(19) United States
(12) Patent Application Publication
(10) Pub. No.: US 2011/0171248 A1
(43) Pub. Date: Jul. 14, 2011

(54) SYNTHETIC VIRUS-LIKE PARTICLES CONJUGATED TO HUMAN PAPILLOMAVIRUS CAPSID PEPTIDES FOR USE AS VACCINES

(75) Inventors: Lynnelle Ann McNamee Pittet, Brighton, MA (US); Yun Gao, Southborough, MA (US); Charles Zepp, Hardwick, MA (US); Grayson B. Lipford, Watertown, MA (US)

(73) Assignee: Selecta Biosciences, Inc., Watertown, MA (US)

(21) Appl. No.: 12/986,874

(22) Filed: Jan. 7, 2011

Related U.S. Application Data
(60) Provisional application No. 61/293,335, filed on Jan. 8, 2010.

(51) Int. Cl. A61K 39/385; C07K 17/00; A61K 38/16; C07K 14/00; C07K 14/025; A61P 31/12; A61P 37/04; C12N 7/00

(52) U.S. Cl. 424/196.11; 530/323; 514/1.1; 530/326; 530/324; 530/350; 435/235.1

(57) ABSTRACT

Disclosed are compositions and related methods that involve a synthetic nanocarrier that includes at least one peptide obtained from Human papillomavirus L1 or L2 capsid protein; wherein the peptide is coupled to an external surface of the synthetic nanocarrier.

### RELATED APPLICATIONS

This application claims the benefit under 35 U.S.C. §119 of U.S. provisional application 61/293,335, filed Jan. 8, 2010, the entire contents of which are incorporated herein by reference.

### BACKGROUND OF THE INVENTION

Human papillomaviruses (HPVs) are small non-enveloped DNA viruses that infect the epidermis and mucous membranes of humans. Over one hundred types of HPV have been identified. About thirty to forty types of HPV can be transmitted through sexual contact. HPV can lead to cancers of the cervix, vulva, vagina, and anus in women. In men, it can lead to cancers of the anus and penis. HPV types 16 and 18 being responsible for approximately 70% of cervical cancers in women worldwide

The HPV virion comprises a ˜8 kb circular dsDNA genome packaged into a ˜60 nm icosahedral capsid made up primarily of two capsid proteins named L1 and L2. The capsid is comprised of 72 pentamers of the major capsid protein L1 and up to 72 molecules of the minor capsid protein L2. Adjacent L1 pentamers are typically crosslinked by disulfide bonds, thus forming the majority of the HPV capsid and stabilizing the capsid structure. The L2 protein is important for HPV infection and is a multifunctional protein, having roles in virion escape from endosomes, genome encapsidation, L1 interaction and capsid stabilization.

HPV infection is a significant public health concern. While there are existing prophylactic vaccines available for prevention of infection, there remains a need for more effective vaccines. Accordingly, what is needed are compositions and methods that provide improved prophylactic treatments against HPV.

### SUMMARY OF THE INVENTION

In an aspect, the invention relates to compositions comprising: a synthetic nanocarrier; at least one peptide obtained from Human papillomavirus L1 or L2 capsid protein; wherein the at least one peptide is coupled to an external surface of the synthetic nanocarrier; and wherein if the at least one peptide obtained from Human papillomavirus L1 or L2 capsid protein comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein.

In an aspect, the invention relates to compounds comprising:

H-Ala-Thr-Gln-Leu-Tyr-Lys-Thr-Cys-Lys-Gln-Ala-Gly-Thr-Cys-Pro-Pro-Asp-Ile-Ile-Pro-Lys-Val-X; [SEQ ID NO. 1]; wherein X is a linker group comprising a terminal alkyne function or an azido function.

In an aspect, the invention relates to compositions comprising: a synthetic nanocarrier; at least one peptide obtained from Human papillomavirus L2 capsid protein, wherein the peptide comprises amino acid residues Cys22 and Cys28 of Human papillomavirus L2 capsid protein; wherein the peptide is coupled to an external surface of the synthetic nanocarrier; and wherein if the at least one peptide obtained from Human papillomavirus L2 capsid protein comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein.

In an aspect, the invention relates to compositions comprising: a synthetic nanocarrier; at least one peptide obtained from Human papillomavirus L1 capsid protein, wherein the peptide comprises a sequence obtained from L1 capsid protein BC loop (aa50-69), DE loop (aa110-153), EF loop (aa160-189), FG loop (aa262-291), or HI loop (aa348-360); or HPV L1 residues 1-173, 111-130, 268-281 or 427-445; and wherein the peptide is coupled to an external surface of the synthetic nanocarrier.

In an aspect, the invention relates to compositions comprising: a synthetic nanocarrier; a universal T-cell antigen; an adjuvant; at least one peptide obtained from Human papillomavirus L2 capsid protein; wherein the at least one peptide is coupled to an external surface of the synthetic nanocarrier; wherein the universal T-cell antigen is coupled to the synthetic nanocarrier; wherein the adjuvant is coupled to the synthetic nanocarrier; and wherein if the at least one peptide obtained from Human papillomavirus L2 capsid protein comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein.

In an aspect, the invention relates to compositions comprising: a synthetic nanocarrier; a universal T-cell antigen; an adjuvant; at least one peptide obtained from Human papillomavirus L1 capsid protein; wherein the at least one peptide is coupled to an external surface of the synthetic nanocarrier; wherein the universal T-cell antigen is coupled to the synthetic nanocarrier; and wherein the adjuvant is coupled to the synthetic nanocarrier.

### BRIEF DESCRIPTION OF THE DRAWINGS

FIG. 1 shows serum anti-L2 peptide antibody titers (EC50) in mice at 26, 40, and 54 days after initial vaccination.

FIG. 2 shows serum anti-L2 peptide antibody titers (EC50) in mice at 139, 153, 156, and 415 days after initial vaccination (additional boost at day 141).

FIG. 3 shows serum anti-L2 peptide antibody titers (EC95) in mice at 26, 40, and 54 days after initial vaccination in comparison to those generated by L2 lipopeptide vaccine (Alphs et al., 2008).

FIG. 4 shows HPV neutralization assay using serum from L2 nanocarrier immunized mice.

FIG. 5 shows serum anti-L2 peptide antibody titers (EC50) in mice at 12, 24, 45, and 59 days after initial vaccination.

FIG. 6 shows serum anti-L2 peptide antibody titers (EC50) in mice at 27, 40, and 54 days after initial vaccination.

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules, reference to “a solvent” includes a mixture of two or more such solvents, reference to “an adhesive” includes mixtures of two or more such materials, and the like.

### A. INTRODUCTION

The inventors have unexpectedly and surprisingly discovered that the problems and limitations noted above can be overcome by practicing the invention disclosed herein. In particular, the inventors have unexpectedly discovered that it is possible to provide compositions, and related methods, comprising: a synthetic nanocarrier; at least one peptide obtained from Human papillomavirus L1 or L2 capsid protein; wherein the at least one peptide is coupled to an external surface of the synthetic nanocarrier and wherein if the at least one peptide obtained from Human papillomavirus L1 or L2 capsid protein comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein.

Also provided is a compound comprising: H-Ala-Thr-Gln-Leu-Tyr-Lys-Thr-Cys-Lys-Gln-Ala-Gly-Thr-Cys-Pro-Pro-Asp-Ile-Ile-Pro-Lys-Val-X; [SEQ ID NO. 1] wherein X is a linker group comprising a terminal alkyne function or an azido function.

Further provided is a composition comprising: a synthetic nanocarrier; at least one peptide obtained from Human papillomavirus L2 capsid protein, wherein the peptide comprises amino acid residues Cys22 and Cys28 of Human papillomavirus L2 capsid protein; wherein the peptide is coupled to an external surface of the synthetic nanocarrier; and wherein if the at least one peptide obtained from Human papillomavirus L2 capsid protein comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein.

Additionally provided is a composition comprising: a synthetic nanocarrier; at least one peptide obtained from Human papillomavirus L1 capsid protein, wherein the peptide comprises a sequence obtained from BC loop (aa50-69), DE loop (aa110-153), EF loop (aa160-189), FG loop (aa262-291), or HI loop (aa348-360); and wherein the peptide is coupled to an external surface of the synthetic nanocarrier.

Provided is a composition comprising: a synthetic nanocarrier; a universal T-cell antigen, an adjuvant; at least one peptide obtained from Human papillomavirus L2 capsid protein; wherein the at least one peptide is coupled to an external surface of the synthetic nanocarrier; wherein the universal T-cell antigen is coupled to the synthetic nanocarrier; wherein the adjuvant is coupled to the synthetic nanocarrier; and wherein if the at least one peptide obtained from Human papillomavirus L2 capsid protein comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein.

Further provided is a composition comprising: a synthetic nanocarrier; a universal T-cell antigen; an adjuvant; at least one peptide obtained from Human papillomavirus L1 capsid protein; wherein the at least one peptide is coupled to an external surface of the synthetic nanocarrier; wherein the universal T-cell antigen is coupled to the synthetic nanocarrier; and wherein the adjuvant is coupled to the synthetic nanocarrier.

The data in the Examples indicate that conjugation of L2 peptide to the surface of nanocarriers leads to significantly improved antibody titers after immunization. Inclusion of the ova peptide in L2 peptide nanocarriers significantly increased the antibody response. Of the two nanocarriers that contained the ova peptide that generated the best anti-L2 antibody titers, the nanocarriers with PLGA-R848 that had a faster R848 release rate (4.6 μg/mg of nanocarriers after 24 hours) generated higher antibody titers than the nanocarriers with PLA-R848 that had a slower R848 release rate (3.4 μg/mg of nanocarriers after 24 hours), even though the PLGA-R848 nanocarriers contained less ova peptide than the PLA-R848 nanocarriers.

These data are supportive of the functionality of the present invention to stimulate an immune response to HPV L2 by administration of the inventive compositions comprising peptides obtained from HPV L2 or modified therefrom. The data also support that a useful immune response to HPV L1 may be stimulated through administration of the inventive compositions comprising peptides obtained from HPV L1 or modified therefrom.

In embodiments wherein an inventive synthetic nanocarrier comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein. This C-terminal coupling strategy, versus coupling through the N-terminal end of the peptide, enhances activity (antibody titer and specificity).

The present invention will now be described in more detail.

### B. DEFINITIONS

“Identity” means the percentage of residues that are identically positioned in a one-dimensional sequence alignment. Identity is a measure of how closely the sequences being compared are related. In an embodiment, identity between two sequences can be determined using the BESTFIT program. In embodiments, the invention pertains to a peptide of 10-30 amino acid residues in length with at least 30%, preferably at least 40%, at least 50%, at least 60%, at least 70%, at least 80%, at least 90%, or at least 95% identity with a peptide from 10-30 amino acid residues in length having a sequence corresponding to a sequence from N terminal amino acids 11-200 of the L2 protein of HPV, wherein the HPV is preferably HPV Type 16 or 18, more preferably HPV Type 16, wherein the identity is as determined using the BESTFIT program.

“Adjuvant” mean an agent that does not constitute a specific antigen, but boosts the immune response to an administered antigen. In specific embodiments, synthetic nanocarriers incorporate as adjuvants compounds that are agonists for toll-like receptors (TLRs) 7 & 8 (“TLR 7/8 agonists”). Of utility are the TLR 7/8 agonist compounds disclosed in U.S. Pat. No. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred adjuvants comprise imiquimod and resiquimod (also known as R848). In specific embodiments, an adjuvant may be an agonist for the DC surface molecule CD40. In certain embodiments, to stimulate immunity rather than tolerance, a synthetic nanocarrier incorporates an adjuvant that promotes DC maturation (needed for priming of naive T cells) and the production of cytokines, such as type I interferons, which promote antibody responses and anti-viral immunity. In embodiments, adjuvants also may comprise immunostimulatory RNA molecules, such as but not limited to dsRNA or poly I:C (a TLR3 stimulant), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an adjuvant may be a TLR-4 agonist, such as bacterial lipopolysacharide (LPS), VSV-G, and/or HMGB-1. In some embodiments, adjuvants may comprise TLR-5 agonists, such as flagellin, or portions or derivatives thereof, including but not limited to those disclosed in U.S. Pat. Nos. 6,130,082, 6,585,980, and 7,192,725. In specific embodiments, synthetic nanocarriers incorporate a ligand for toll-like receptor (TLR)-9, such as CpGs, which induce type I interferon production. In some embodiments, adjuvants may be proinflammatory stimuli released from necrotic cells (e.g., urate crystals). In some embodiments, adjuvants may be activated components of the complement cascade (e.g., CD21, CD35, etc.). In some embodiments, adjuvants may be activated components of immune complexes. The adjuvants also include complement receptor agonists, such as a molecule that binds to CD21 or CD35. In some embodiments, the complement receptor agonist induces endogenous complement opsonization of the synthetic nanocarrier. In some embodiments, adjuvants are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer. In embodiments, adjuvants may be coupled to synthetic nanocarriers. In other embodiments, adjuvants may be uncoupled from the synthetic nanocarriers.

“Administering” or “administration” means providing a drug to a subject in a manner that is pharmacologically useful.

“Antigen” means a B cell antigen or T cell antigen.

“Couple” or “Coupled” or “Couples” (and the like) means to chemically associate one entity (for example a moiety) with another. In some embodiments, the coupling is covalent, meaning that the coupling occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of coupling.

“Dosage form” means a drug in a medium, carrier, vehicle, or device suitable for administration to a subject.

“Encapsulate” means to enclose within a synthetic nanocarrier, preferably enclose completely within a synthetic nanocarrier. Most or all of a substance that is encapsulated is not exposed to the local environment external to the synthetic nanocarrier. Encapsulation is distinct from absorption, which places most or all of a substance on a surface of a synthetic nanocarrier, and leaves the substance exposed to the local environment external to the synthetic nanocarrier.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheriodal synthetic nanocarrier, the maximum and minimum dimension of a synthetic nanocarrier would be substantially identical, and would be the size of its diameter. Similarly, for a cubic synthetic nanocarrier, the minimum dimension of a synthetic nanocarrier would be the smallest of its height, width or length, while the maximum dimension of a synthetic nanocarrier would be the largest of its height, width or length.

In an embodiment, a minimum dimension of at least 75%, preferably at least 80%, more preferably at least 90%, of the synthetic nanocarriers in a sample, based on the total number of synthetic nanocarriers in the sample, is greater than 100 nm. In a embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a maximum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier sizes is obtained by suspending the synthetic nanocarriers in a liquid (usually aqueous) media and using dynamic light scattering (e.g. using a Brookhaven ZetaPALS instrument).

“Release” or “Release Rate” means the rate that an entrapped substance transfers from a synthetic nanocarrier into local environment, such as a surrounding release media. First, the synthetic nanocarrier is prepared for the release testing by placing into the appropriate release media. This is generally done by exchanging a buffer after centrifugation to pellet the synthetic nanocarrier and reconstitution of the synthetic nanocarriers under a mild condition. The assay is started by placing the sample at 37° C. in an appropriate temperature-controlled apparatus. A sample is removed at various time points.

The synthetic nanocarriers are separated from the release media by centrifugation to pellet the synthetic nanocarriers. The release media is assayed for the substance that has been released from the synthetic nanocarriers. The substance is measured using HPLC to determine the content and quality of the substance. The pellet containing the remaining entrapped substance is dissolved in solvents or hydrolyzed by base to free the entrapped substance from the synthetic nanocarriers. The pellet-containing substance is then also measured by HPLC to determine the content and quality of the substance that has not been released at a given time point.

The mass balance is closed between substance that has been released into the release media and what remains in the synthetic nanocarriers. Data is presented as the fraction released or as the net release presented as micrograms released over time.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, inventive synthetic nanocarriers do not comprise chitosan.

A synthetic nanocarrier can be, but is not limited to, one or a plurality of lipid-based nanoparticles (e.g. liposomes), polymeric nanoparticles, metallic nanoparticles, surfactant-based emulsions, dendrimers, buckyballs, nanowires, virus-like particles, peptide or protein-based particles (such as albumin nanoparticles) and/or nanoparticles that are developed using a combination of nanomaterials such as lipid-polymer nanoparticles. Synthetic nanocarriers may be a variety of different shapes, including but not limited to spheroidal, cubic, pyramidal, oblong, cylindrical, toroidal, and the like. Synthetic nanocarriers according to the invention comprise one or more surfaces, including but not limited to internal surfaces (surfaces generally facing an interior portion of the synthetic nanocarrier) and external surfaces (surfaces generally facing an external environment of the synthetic nanocarrier). Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (4) the lithographically constructed nanoparticles of Published US Patent Application 20090028910 to DeSimone et al., (5) the disclosure of WO 2009/051837 to von Andrian et al., or (6) the nanoparticles disclosed in Published US Patent Application 2008/0145441 to Penades et al.

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers may possess an aspect ratio greater than 1:1, 1:1.2, 1:1.5, 1:2, 1:3, 1:5, 1:7, or greater than 1:10.

“T cell antigen” means any antigen that is recognized by and triggers an immune response in a T cell (e.g., an antigen that is specifically recognized by a T cell receptor on a T cell or an NKT cell via presentation of the antigen or portion thereof bound to a Class I or Class II major histocompatability complex molecule (MHC), or bound to a CD1 complex. In some embodiments, an antigen that is a T cell antigen is also a B cell antigen. In other embodiments, the T cell antigen is not also a B cell antigen. T cell antigens generally are proteins or peptides. T cell antigens may be an antigen that stimulates a CD8+ T cell response, a CD4+ T cell response, or both. The T cell antigens, therefore, in some embodiments can effectively stimulate both types of responses. In some embodiments the CD4+ T cell antigen is a “Universal T cell antigen,” meaning a T cell antigen that can generate an enhanced response to an unrelated B cell antigen through stimulation of T cell help. In embodiments, a universal T cell antigen may comprise one or more peptides obtained from tetanus toxoid, Epstein-Barr virus, influenza virus, respiratory syncytial virus, cytomegalovirus, adenovirus, diphtheria toxoid, or a PADRE peptide. In other embodiments, a universal T cell antigen may comprise one or more lipids, or glycolipids, including but not limited to: α-galactosylceramide (α-GalCer), α-linked glycosphingolipids (from Sphingomonas spp.), galactosyl diacylglycerols (from Borrelia burgdorferi), lypophosphoglycan (from Leishmania donovani), and phosphatidylinositol tetramannoside (PIM4) (from Mycobacterium leprae). For additional lipids and/or glycolipids useful as universal T cell antigens, see V. Cerundolo et al., “Harnessing invariant NKT cells in vaccination strategies.” Nature Rev Immun, 9:28-38 (2009). In embodiments, CD4+ T-cell antigens may be derivatives of a CD4+ T-cell antigen that is obtained from a source, such as a natural source. In such embodiments, CD4+ T-cell antigen sequences, such as those peptides that bind to MHC II, may have at least 70%, 80%, 90%, or 95% identity to the antigen obtained from the source. In embodiments, the T cell antigen, preferably the universal T cell antigen, may be coupled to, or uncoupled from, a synthetic nanocarrier.

### C. INVENTIVE IMMUNONANOTHERAPEUTIC COMPOSITIONS

HPV capsid proteins L1 and L2 include a number of conserved regions, some of which are suitable for use as neutralizing epitopes across multiple types of HPV.

In an embodiments, at least one peptide obtained from HPV L2 capsid protein can be useful in creating peptide epitopes that can be incorporated into vaccines according to the invention. In other embodiments, a peptide from 10-30 amino acid residues in length having a sequence corresponding to a sequence from N terminal amino acids 11-200 of Human papillomavirus L2 protein can be incorporated into inventive vaccines. In still other embodiments, peptides can be used that comprise amino acid residues 15-36 of the HPV L2 capsid protein. In certain embodiments, the peptide comprises: H-Ala-Thr-Gln-Leu-Tyr-Lys-Thr-Cys-Lys-Gln-Ala-Gly-Thr-Cys-Pro-Pro-Asp-Ile-Ile-Pro-Lys-Val-NH2 (SEQ ID NO 2). In other embodiments, the peptide comprises amino acid residues Cys22 and Cys28 of Human papillomavirus L2 capsid protein. See S. Campos, et al., PLos-One, 2009, 4: e4463. In certain embodiments, the peptide comprises amino acid residues Cys22 and Cys28 of Human papillomavirus L2 capsid protein wherein a disulfide bond is present between amino acid residues Cys22 and Cys28.

Additional information about regions and sequences from L1 and L2 that could be useful in the practice of the present invention may be found, for example, in J. Lowe et al., “Evolutionary and structural analyses of alpha-papillomavirus capsid proteins yields novel insights into L2 structure and interaction with L1.” Virology Journal 5:150 (2008); US Patent Application 20090047301, entitled Papillomavirus L2 N-Terminal Peptides for the Induction of Broadly Cross-Neutralizing Antibodies; U.S. Pat. No. 6,599,739 entitled Infectious papillomavirus pseudoviral particles, U.S. Pat. No. 6,174,532 entitled L2 immunogenic peptides of papillomavirus; H. Alphs et al. “Protection against heterologous human papillomavirus challenge by a synthetic lipopeptide vaccine containing a broadly cross-neutralizing epitope of L2.” PNAS. 2008 Apr. 15; 105(15):5850-5; S. Campos et al., “Two highly conserved cysteine residues in HPV16 L2 form an intramolecular disulfide bond and are critical for infectivity in human keratinocytes.” PLoS One. 2009; 4(2):e4463; P. Day et al., “Mechanisms of human papillomavirus type 16 neutralization by L2 cross-neutralizing and L1 type-specific antibodies.” J. Virology. 2008; 82(9):4638-4646; R. Gambhira et al., “A protective and broadly cross-neutralizing epitope of human papillomavirus L2”. J. Virology. 2007; 81(24)13927-13931; and S. Jagu et al. “Concatenated multitype L2 fusion proteins as candidate prophylactic pan-human papillomavirus vaccines.” JNCI. 2009; 101(11):782-792. Further sequences useful as peptides of the present invention are found in Table 4.

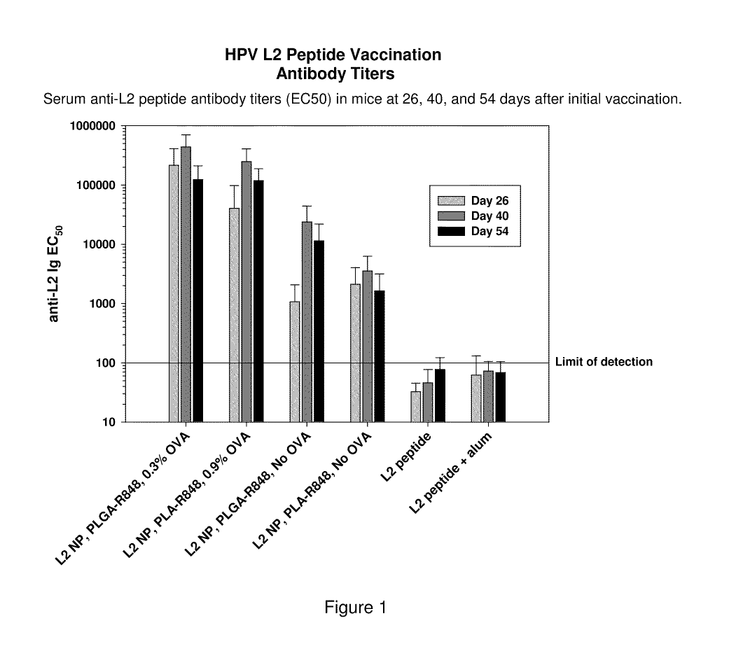

In certain embodiments, at least one peptide obtained from HPV L1 capsid protein can be useful in creating peptide epitopes that can be incorporated into vaccines according to the invention. In embodiments, useful peptides comprise those L1 peptides obtained from L1 capsid protein BC loop (aa50-69), DE loop (aa110-153), EF loop (aa160-189), FG loop (aa262-291), or HI loop (aa348-360), or HPV L1 residues 1-173, 111-130, 268-281 or 427-445. Information about the BC, DE, EF, FG, and HI loop sequences can be found in FIG. 1 of Bishop B. et al., “Crystal Structures of Four Types of Human Papillomavirus L1 Capsid Proteins.” J Biol. Chem. 2007. 282(43):31803-31811. Information about sequences for HPV L1 residues 1-173, 111-130, 268-281 or 427-445 can be found in Table 4 or in the following articles: Carter et al., “Identification of a Human Papillomavirus Type 16-Specific Epitope on the C-Terminal Arm of the Major Capsid Protein L1.” J. Virol. 2003. 77(21):11625-11632; and Christensen et al., “Hybrid Papillomavirus L1 Molecules Assemble into Virus-like Particles That Reconstitute Conformational Epitopes and Induce Neutralizing Antibodies to Distinct HPV Types.” Virology. 2001.291(2):324-334.

In various embodiments, the recited peptides can be obtained from L1 or L2 proteins obtained from various types of HPV types comprising types 16, 18, 26, 31, 33, 35, 39, 45, 51, 52, 53, 56, 58, 59, 66, 68, 73, or 82, more preferably types 16 or 18, and most preferably type 16.

The peptides may be modified from a peptide originally obtained from an L1 or L2 protein. In an embodiments, the invention encompasses a peptide of 10-200, preferably 10-100, more preferably 10-30 amino acid residues in length with at least 30%, at least 40%, at least 50%, at least 60%, at least 70%, at least 80%, at least 90%, or at least 95% identity to a sequence from a peptide originally obtained from an L1 or L2 protein, with identity determined using the BESTFIT program. In an embodiment, the peptide comprises from 10-30 amino acid residues in length having at least 30%, at least 40%, at least 50%, at least 60%, at least 70%, at least 80%, at least 90%, or at least 95% identity to a sequence obtained from N terminal amino acids 11-200 of HPV L2 protein, as determined using the BESTFIT program.

Peptides can be coupled to the synthetic nanocarriers by a variety of methods. In embodiments, the peptide is coupled to an external surface of the synthetic nanocarrier covalently or non-covalently, preferably though its C terminus or its N-terminus, more preferably the peptide is coupled through its C terminus to the external surface.

In certain embodiments, the coupling can be a covalent linker. In embodiments, peptides according to the invention can be covalently coupled to the external surface via a 1,2,3-triazole linker formed by the 1,3-dipolar cycloaddition reaction of azido groups on the surface of the nanocarrier with peptides containing an alkyne group or by the 1,3-dipolar cycloaddition reaction of alkynes on the surface of the nanocarrier with peptides containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, the covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component such as the peptide with the carboxylic acid group of a second component such as the nanocarrier. The amide bond in the linker can be made using any of the conventional amide bond forming reactions with suitably protected amino acids or peptides and activated carboxylic acid such N-hydroxysuccinimide-activated ester.

A disulfide linker is made via the formation of disulfide (S—S) bond between two sulfur atoms of the form, for instance, of R₁—S—S—R₂. A disulfide bond can be formed by thiol exchange of a peptides containing thiol/mercaptan group(—SH) with another activated thiol group on a polymer or nanocarrier or a nanocarrier containing thiol/mercaptan groups with a peptide containing activated thiol group.

A triazole linker, specifically a 1,2,3-triazole of the form

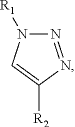

wherein R₁ and R₂ may be any chemical entities, is made by the 1,3-dipolar cycloaddition reaction of an azide attached to a first component such as the nanocarrier with a terminal alkyne attached to a second component such as the peptide. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as “click” reaction or CuAAC.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The peptide is prepared with the presence of either an alkyne (if the polymer contains an azide) or an azide (if the polymer contains an alkyne) group. The antigen is then allowed to react with the nanocarrier via the 1,3-dipolar cycloaddition reaction with or without a catalyst which covalently couples the antigen to the particle through the 1,4-disubstituted 1,2,3-triazole linker.

A thioether linker is made by the formation of a sulfur-carbon (thioether) bond in the form, for instance, of R₁—S—R₂. Thioether can be made by either alkylation of a thiol/mercaptan (—SH) group on one component such as the peptide with an alkylating group such as halide or epoxide on a second component such as the nanocarrier. Thioether linkers can also be formed by Michael addition of a thiol/mercaptan group on one component such as a peptide to an electron-deficient alkene group on a second component such as a polymer containing a maleimide group as the Michael acceptor. In another way, thioether linkers can be prepared by the radical thiol-ene reaction of a thiol/mercaptan group on one component such as a peptide with an alkene group on a second component such as a polymer or nanocarrier.

A hydrazone linker is made by the reaction of a hydrazide group on one component such as the peptide with an aldehyde/ketone group on the second component such as the nanocarrier.

A hydrazide linker is formed by the reaction of a hydrazine group on one component such as the peptide with a carboxylic acid group on the second component such as the nanocarrier. Such reaction is generally performed using chemistry similar to the formation of amide bond where the carboxylic acid is activated with an activating reagent.

An imine or oxime linker is formed by the reaction of an amine or N-hydroxylamine group on one component such as the peptide with an aldehyde or ketone group on the second component such as the nanocarrier.

An urea or thiourea linker is prepared by the reaction of an amine group on one component such as the peptide with an isocyanate or thioisocyanate group on the second component such as the nanocarrier.

An amidine linker is prepared by the reaction of an amine group on one component such as the peptide with an imidoester group on the second component such as the nanocarrier.

An amine linker is made by the alkylation reaction of an amine group on one component such as the peptide with an alkylating group such as halide, epoxide, or sulfonate ester group on the second component such as the nanocarrier. Alternatively, an amine linker can also be made by reductive amination of an amine group on one component such as the peptide with an aldehyde or ketone group on the second component such as the nanocarrier with a suitable reducing reagent such as sodium cyanoborohydride or sodium triacetoxyborohydride.

A sulfonamide linker is made by the reaction of an amine group on one component such as the peptide with an sulfonyl halide (such as sulfonyl chloride) group on the second component such as the nanocarrier.

Additional descriptions of available conjugation methods are available in “Bioconjugate Techniques”, 2nd Edition By Greg T. Hermanson, Published by Academic Press, Inc., 2008.

In embodiments, certain of the peptide's amino acid residues may be residues that have been chemically modified to promote coupling. While such amino acid residues may be chemically modified before or after the peptide has been formed, it is presently preferred to use chemically modified amino acids when synthesizing the peptides using chemical synthesis techniques. A preferred modified peptide comprises: H-Ala-Thr-Gln-Leu-Tyr-Lys-Thr-Cys-Lys-Gln-Ala-Gly-Thr-Cys-Pro-Pro-Asp-Ile-Ile-Pro-Lys-Val-X (SEQ ID NO. 1); wherein X is a linker group comprising a terminal alkyne group or an azido group. In preferred embodiments, (a) the terminal alkyne group comprises propargyl NH—CH2CCH (triple bond between CCH), Lys(5-hexynoyl)-NH2, or Gly-propargylamide; or (b) wherein the azido group comprises NH—(CH2)n-N3 where n>=2, or Lys(6-N3)-OH, or Lys(6-N3)-NH2. In an embodiments, the peptide comprises the peptide of SEQ ID NO. 1, wherein X comprises Lys(5-hexynoyl)-NH2 (SEQ ID NO: 3).

The peptide can also be conjugated to the nanocarrier via non-covalent conjugation methods. For examples, a negative charged peptide can be conjugated to a positive charged nanocarrier through electrostatic adsorption. A peptide containing a metal ligand can also be conjugated to a nanocarrier containing a metal complex via a metal-ligand complex.

In embodiments, compositions according to the invention may comprise more than one peptide obtained from Human papillomavirus L1 or L2 capsid protein coupled to an external surface of the synthetic nanocarrier. Coupling multiple copies of the peptide to an external surface of a synthetic nanocarrier is preferred because it may enhance the immunogenicity of the synthetic nanocarrier that is so produced.

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

It is often desirable to use a population of synthetic nanocarriers that is relatively uniform in terms of size, shape, and/or composition so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers. In some embodiments, a population of synthetic nanocarriers may be heterogeneous with respect to size, shape, and/or composition.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In some embodiments, synthetic nanocarriers can comprise one or more polymeric matrices. In some embodiments, such a polymeric matrix can be surrounded by a coating layer (e.g., liposome, lipid monolayer, micelle, etc.). In some embodiments, various elements of the synthetic nanocarriers can be coupled with the polymeric matrix.

In some embodiments, an immunofeature surface, targeting moiety, and/or oligonucleotide can be covalently associated with a polymeric matrix. In some embodiments, covalent association is mediated by a linker. In some embodiments, an immunofeature surface, targeting moiety, and/or oligonucleotide can be noncovalently associated with a polymeric matrix. For example, in some embodiments, an immunofeature surface, targeting moiety, and/or oligonucleotide can be encapsulated within, surrounded by, and/or dispersed throughout a polymeric matrix. Alternatively or additionally, an immunofeature surface, targeting moiety, and/or nucleotide can be associated with a polymeric matrix by hydrophobic interactions, charge interactions, van der Waals forces, etc.

A wide variety of polymers and methods for forming polymeric matrices therefrom are known in the art of drug delivery. In general, a polymeric matrix comprises one or more polymers. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

Examples of polymers suitable for use in the present invention include, but are not limited to polyethylenes, polycarbonates (e.g. poly(1,3-dioxan-2one)), polyanhydrides (e.g. poly(sebacic anhydride)), polyhydroxyacids (e.g. poly(β-hydroxyalkanoate)), polypropylfumerates, polycaprolactones, polyamides (e.g. polycaprolactam), polyacetals, polyethers, polyesters (e.g., polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines.

In some embodiments, polymers in accordance with the present invention include polymers which have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g. coupled) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, polyanhydrides, poly(ortho ester), poly(ortho ester)-PEG copolymers, poly(caprolactone), poly(caprolactone)-PEG copolymers, polylysine, polylysine-PEG copolymers, poly(ethyleneimine), poly(ethylene imine)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines. In embodiments, the inventive synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-Llysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-praline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that inventive synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers may not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such assorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In certain embodiments, the carbohydrate is a sugar alcohol, including but not limited to mannitol, sorbitol, xylitol, erythritol, maltitol, and lactitol. In embodiments, the inventive synthetic nanocarriers do not comprise (or specifically exclude) carbohydrates, such as a polysaccharide.

Compositions according to the invention comprise inventive synthetic nanocarriers in combination with pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, inventive synthetic nanocarriers are suspended in sterile saline solution for injection together with a preservative.

### D. METHODS OF MAKING AND USING THE INVENTIVE IMMUNONANOTHERAPEUTICS

Synthetic nanocarriers may be prepared using a wide variety of methods known in the art. For example, synthetic nanocarriers can be formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755, and also U.S. Pat. Nos. 5,578,325 and 6,007,845).

Various materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006). Other methods suitable for encapsulating materials, such as oligonucleotides, into synthetic nanocarriers may be used, including without limitation methods disclosed in U.S. Pat. No. 6,632,671 to Unger Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be coupled to the synthetic nanocarriers and/or the composition of the polymer matrix.

If particles prepared by any of the above methods have a size range outside of the desired range, particles can be sized, for example, using a sieve.

Elements of the inventive synthetic nanocarriers besides the peptides obtained from HPV L1 or L2 proteins, or derived from them—such as moieties of which an immunofeature surface is comprised, targeting moieties, polymeric matrices, and the like—may be coupled to the synthetic nanocarrier, e.g., by one or more covalent bonds, or may be coupled by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be coupled to immunofeature surfaces, targeting moieties, adjuvants, and/or other elements directly or indirectly via non-covalent interactions. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on an external surface or an internal surface of an inventive synthetic nanocarrier. In embodiments, encapsulation is a form of coupling.

It is to be understood that the compositions of the invention can be made in any suitable manner, and the invention is in no way limited to compositions that can be produced using the methods described herein. Selection of an appropriate method may require attention to the properties of the particular moieties being associated.

In some embodiments, inventive synthetic nanocarriers are manufactured under sterile conditions or are terminally sterilized. This can ensure that resulting composition are sterile and non-infectious, thus improving safety when compared to non-sterile compositions. This provides a valuable safety measure, especially when subjects receiving synthetic nanocarriers have immune defects, are suffering from infection, and/or are susceptible to infection. In some embodiments, inventive synthetic nanocarriers may be lyophilized and stored in suspension or as lyophilized powder depending on the formulation strategy for extended periods without losing activity.

The inventive compositions may be administered by a variety of routes of administration, including but not limited to parenteral (such as subcutaneous, intramuscular, intravenous, or intradermal); oral; transnasal, transmucosal, rectal; ophthalmic, or transdermal.

The compositions and methods described herein can be used to induce, enhance, suppress, direct, or redirect an immune response. The compositions and methods described herein can be used for the prophylaxis and/or treatment of conditions such as cancers, infectious diseases, metabolic diseases, degenerative diseases, autoimmune diseases, inflammatory diseases, immunological diseases, or other disorders and/or conditions. The compositions and methods described herein can also be used for the prophylaxis and/or treatment of a condition resulting from a harmful agent.

### E. EXAMPLES

### Example 1

### Preparation of PLGA-R848

PLGA-R848 was prepared by reaction of PLGA polymer containing acid end group with R848 in the presence of coupling agent such as HBTU as follows:

A mixture of PLGA (Lakeshores Polymers, MW˜5000, 7525DLG1A, acid number 0.7 mmol/g, 10 g, 7.0 mmol) and HBTU (5.3 g, 14 mmol) in anhydrous EtOAc (160 mL) was stirred at room temperature under argon for 50 minutes. Compound R848 (2.2 g, 7 mmol) was added, followed by diisopropylethylamine (DIPEA) (5 mL, 28 mmol). The mixture was stirred at room temperature for 6 h and then at 50-55° C. overnight (about 16 h). After cooling, the mixture was diluted with EtOAc (200 mL) and washed with saturated NH₄Cl solution (2×40 mL), water (40 mL) and brine solution (40 mL). The solution was dried over Na₂SO₄ (20 g) and concentrated to a gel-like residue. Isopropyl alcohol (IPA) (300 mL) was then added and the polymer conjugate precipitated out of solution. The polymer was then washed with IPA (4×50 mL) to remove residual reagents and dried under vacuum at 35-40° C. for 3 days as a white powder (10.26 g, MW by GPC was 5200, R848 loading was 12% by HPLC).

In a similar manner, PLA-R848 was prepared by the reaction of PLA-CO2H (polylactide with acid ending group) with R848.

### Example 2

### Preparation of PLA-PEG-PEG3-N3 Copolymer

PLA-PEG-N3 polymer was prepared by ring opening polymerization of HO-PEG-azide with dl-lactide in the presence of Sn(Oct)₂ catalyst as follows:

HO-PEG-CO2H (MW 3500, 1.33 g, 0.38 mmol) was treated with NH2-PEG3-N3 (MW 218.2, 0.1 g, 0.458 mmol) in the presence of DCC (MW 206, 0.117 g, 0.57 mmol) and NHS (MW 115, 0.066 g, 0.57 mmol) in dry DCM (10 mL) overnight. After filtration to remove insoluble byproduct (DCC-urea), the solution was concentrated and then diluted with ether to precipitate out the polymer, HO-PEG-PEG3-N3 (1.17 g). After drying, HO-PEG-PEG3-N3 (MW 3700, 1.17 g, 0.32 mmol) was mixed with dl-lactide (recrystallized from EtOAc, MW 144, 6.83 g, 47.4 mmol) and Na2SO4 (10 g) in a 100 mL flask. The solid mixture was dried under vacuum at 45 C overnight and dry toluene (30 mL) was added. The resulting suspension was heated to 110° C. under argon and Sn(Oct)2 (MW 405, 0.1 mL, 0.32 mmol) was added. The mixture was heated at reflux for 18 h and cooled to rt. The mixture was diluted with DCM (50 mL) and filtered. After concentration to an oily residue, MTBE (200 mL) was added to precipitate out the polymer which was washed once with 100 mL of 10% MeOH in MTBE and 50 mL of MTBE. After drying, PLA-PEG-PEG3-N3 was obtained as a white foam (7.2 g, average MW: 23,700 by H NMR).

### Example 3

### Prep of PLA-PEG-C6-N3 Copolymer

HO-PEG-CO2H (MW 3500, 1.00 g, 0.29 mmol) was treated with 6-azido-1-hexylamine (H2N—C6-N3) (MW 142, 0.081 g, 0.57 mmol) in the presence of DCC (MW 206, 0.118 g, 0.57 mmol) and NHS (MW 115, 0.066 g, 0.57 mmol) in dry DCM (10 mL) overnight. After filtration to remove insoluble byproduct (DCC-urea), the solution was concentrated and then diluted with MTBE to precipitate out the polymer which was then washed twice with MTBE and dried under vacuum at 30 C overnight to give HO-PEG-C6-N3 polymer (1.1 g).

HO-PEG-C6-N3 polymer (1.1 g, 0.29 mmol) and dl-lactide (6.5 g, 45 mmol) were mixed in dry toluene (60 mL). The mixture was heated to reflux while 30 mL of toluene was removed by azeotrope distillation. The resulting solution was cooled to 100° C. and Sn(Oct)2 (0.095 mL, 0.29 mmol) was added. The solution was heated at reflux under argon overnight and cooled to room temperature (i.e. “rt”). The solution was then added to 150 mL of 2-propanol to precipitate out the polymer which was washed with 2-propanol (100 mL) and dried under vacuum at 30° C. for 2 days to give PLA-PEG-C6-N3 copolymer as an off-white solid (6.8 g, MW by GPC is 27000 with DPI of 1.5).

### Example 4

### PLA-PEG(5K)-CONH2NH2 Copolymer

A mixture of HO-PEG(5k)-CO2H (JenKem Technology, USA) (MW: 5000, 1.0 g, 0.2 mmol), tert-butyl carbazate (Boc-hydrazide) (MW: 132, 0.053 g, 0.4 mmol), DCC (MW 206, 0.083 g, 0.4 mmol) and N-hydroxysuccinimide (NHS) (MW 115, 0.05 g, 0.4 mmol) in dry DCM (15 mL) was stirred at rt for 25 h. The insoluble DCC-urea was removed by filtration and the filtrate was concentrated. The residual was added to 50 mL of MTBE to precipitate out the polymer which was washed twice with 40 mL of MTBE and dried under vacuum for 2 days to give HO-PEG(5k)-CONHNHtBoc as a white powder (1.07 g).

HO-PEG(5k)-CONHNHtBoc polymer (1.07 g, 0.20 mmol) and dl-lactide (4.32 g, 30 mmol) were mixed in dry toluene (70 mL). The mixture was heated to reflux while 50 mL of toluene was removed by azeotrope distillation. The resulting solution was cooled to 100° C. and Sn(Oct)₂ (0.065 mL, 0.20 mmol) was added. The solution was heated at reflux under argon for 22 h and cooled to rt. The solution was then added to 150 mL of 2-propanol to precipitate out the polymer which was washed with 2-propanol (60 mL) and dried under vacuum at 30° C. for 2 days to give PLA-PEG(5k)-CONHNHtBoc copolymer as a white solid chunk. The polymer was dissolved in 50 mL of dry DCM and cooled with ice water. Trifluoroacetic acid (TFA) (15 mL) was added and the resulting solution was stirred at rt overnight. The yellowish solution was concentrated to dryness. The residual was added to 200 mL of 2-propanol to precipitate out the polymer which was washed with 100 mL of 2-propanol. The polymer was dried at 30° C. under vacuum to give the desired polymer as PLA-PEG(5k)-CONHNH2 (3.4 g, MW by NMR: 24000).

### Example 5

### PLA-PEG-MAL

HO-PEG(3K)-maleimide (HO-PEG-MAL) (Laysan Bio, Inc) (MW: 3000, 0.6 g, 0.2 mmol) was mixed with dl-lactide (recrystallized from EtOAc, MW 144, 4.32 g, 30 mmol) and Na₂SO₄ (4 g) in a 100 mL flask. The solid mixture was dried under vacuum at 60° C. overnight and dry toluene (20 mL) was added. The resulting suspension was heated to 110° C. under argon and Sn(Oct)₂ (MW 405, 0.065 mL, 0.2 mmol) was added. The mixture was heated at reflux for 20 h and cooled to rt. The mixture was diluted with DCM (50 mL) and filtered. After concentration to an oily residue, 10% MeOH in ethyl ether (80 mL) was added to precipitate out the polymer which was washed once with 80 mL of 10% MeOH in ether and 60 mL of ether. After drying at 30° C. under vacuum overnight, PLA-PEG(3K)-MAL was obtained as a white foam (3.26 g, average MW: 24,000 by H NMR).

### Example 6

### Preparation of PLA-PEG-SH (Prophetic)

PLA-PEG-SH copolymer is prepared according to the literature procedure (Nisha C. Kalarickal, et al; Macromolecules 2007, 40:1874-1880).

Step-1. Preparation of tBuS-PEG: Anhydrous THF (22 mL), potassium naphthalene (0.2 M solution in THF, 12 mL), and tBu-SH (0.54 mL, 4.8 mmol) are charged into a sealed 100 mL round-bottom flask. The components are stirred for at least 15 min to ensure the formation of thiolates, at which point liquid ethylene oxide (EO) (11.5 mL, 0.230 mol) is added using a two-head needle. The polymerization reaction is carried out for 48 h, and the product is recovered by precipitation in cold diethyl ether. MW of the polymer by GPC is about 2100.

Step-2. Preparation of (PEG-S)2: tBu-S-PEG from Step-1 (1.0 g) is dissolved in DMSO (19 mL) followed by addition of TFA (106 mL, 15/85 v/v) to a final polymer concentration of 8 mg/mL. The reaction is stirred for 20 min, after which TFA is removed by rotary evaporation. The residual is then precipitated twice in cold diethyl ether to recover the crude PEG disulfide. The crude (PEG-S)2 is further purified by fractional precipitation. Thus, the polymer (1.0 g) is dissolved in dichloromethane (100 mL), and then cold diethyl ether is added stepwise with stirring until the appearance of a precipitate. The solution is further stirred for 30 min, and the precipitated mass is isolated by filtration and dried in vacuum. The recovery yield of PEG disulfide, (PEG-S)₂, at the end of two to three fractional precipitations is in the range 55-60%.

Step-3. Preparation of (PLA-b-PEG-S)2 by ring-opening polymerization of dl-lactide: (PEG-S)2 (0.4 g, 0.10 mmol) and dl-lactide (4.32 g, 30 mmol) are mixed in dry toluene (70 mL). The mixture is heated to reflux while 50 mL of toluene is removed by azeotrope distillation. The resulting solution is cooled to 100 C and Sn(Oct)2 (0.065 mL, 0.20 mmol) is added. The solution is heated at reflux under argon for 18-20 h and cooled to rt. The solution is then added to 150 mL of 2-propanol to precipitate out the polymer which is washed with 2-propanol (60 mL) and ether (60 mL) and dried under vacuum at 30 C for 2 days to give (PLA-PEG-S)2 (ca. 4.0 g, MW: 46000).

Step-4. Preparation of PLA-PEG-SH by Reduction of (PLA-PEG-S)2:

The (PLA-PEG-S)2 from Step-3 (3.2 g, 0.07 mmol) is dissolved in deoxygenated THF (25 mL), and Bu3P (1.7 mL, 7.0 mmol, 100 equiv with respect to disulfide units) is added. The reaction mixture is stirred under argon at room temperature overnight. The reduced thiolated polymer is recovered by precipitation in cold diethyl ether followed by filtration under argon atmosphere and further dried under vacuum to give PLA-PEG-SH as an off white chunky solid (ca. 3.0, MW: 23000)

### Example 7a

### General Procedure for the Preparation of Nanocarriers (NCs) with Surface PEG-X from PLA-PEG-X where X=C6-azide (C6-N3) or PEG3-azide (PEG3-N3) and PLGA-R848/PLA-R848 with T-Cell Antigen, Ova Peptide

Nanocarriers comprising PLGA-R848 or PLA-R848, PLA-PEG-X, and ova peptide were prepared via double emulsion method wherein the ova peptide was encapsulated in the nanocarriers.

The polyvinyl alcohol (Mw=11 KD-31 KD, 87-89% partially hydrolyzed) was purchased from JT Baker. Ovalbumin peptide 323-339, (sequence: H-Ile-Ser-Gln-Ala-Val-His-Ala-Ala-His-Ala-Gludle-Asn-Glu-Ala-Gly-Arg-NH2 (SEQ ID NO: 4), acetate salt, Lot# B06395) was obtained from Bachem Americas Inc. (3132 Kashiwa Street, Torrance Calif. 90505), PLA-CO2H (100DL2A) was obtained from SurModics Pharmaceuticals

(756 Tom Martin Drive, Birmingham, Ala. 35211), PLGA-R848 or PLA-R848, and PLA-PEG-X conjugates were prepared as described in above examples.

The above materials were used to prepare the following solutions:

1. PLGA-R848 or PLA-R848 conjugate in methylene chloride @ 100 mg/mL

2. PLA-PEG-X in methylene chloride @ 100 mg/mL

3. PLA-CO2H in methylene chloride @ 100 mg/mL

4. Ovalbumin peptide 323-339 in 0.13N HCl @ 70 mg/mL

5. Polyvinyl alcohol in 100 mM pH 8 phosphate buffer @50 mg/mL

Solution #1 (0.50 mL), solution #2 (0.25 mL) and solution #3 (0.25 mL) were combined and solution #4 in 0.13N HCl (0.1 mL) was added in a small vessel and the mixture was sonicated at 50% amplitude for 40 seconds using a Branson Digital Sonifier 250. To this emulsion was added solution # 5 (2.0 mL) and sonication at 30% amplitude for 40 seconds using the Branson Digital Sonifier 250 to the second emulsion.

This was added to a stirring beaker containing a 70 mM pH 8 phosphate buffer solution (30 mL), and this mixture was stirred at room temperature for 2 hours to form the nanocarriers.

To wash the nanocarriers, a portion of the nanoparticle dispersion (26.5 mL) was transferred to a 50 mL centrifuge tube and spun at 9500 rpm (13,800 g) for one hour at 4° C., the supernatant was removed, and the pellet was re-suspended in 26.5 mL of phosphate buffered saline. The centrifuge procedure was repeated and the pellet was re-suspended in 8.3 g of phosphate buffered saline for a final nanocarrier dispersion of about 10 mg/mL containing encapsulated ova peptide.

In a similar manner, nanocarrier without ova peptide was also prepared where solution #4 was eliminated in the preparation.

In a similar manner, nanocarrier without PLGA-R848 or PLA-R848 was also prepared where solution #1 was eliminated in the preparation.

### Example 7b

### General procedure for the Preparation of Nanocarriers (NCs) with Surface PEG-X from PLA-PEG-X where X=Hydrazide (CONHNH2), Maleimide (MAL), Thiol (SH) and PLGA-R848/PLA-R-848 with T-Cell Antigen, Ova Peptide (Prophetic)

Nanocarriers comprising PLGA-R848 or PLA-R848, PLA-PEG-X, and ova peptide are prepared via double emulsion method wherein the ova peptide is encapsulated in the nanocarriers.

The polyvinyl alcohol (Mw=11 KD-31 KD, 87-89% partially hydrolyzed) is purchased from JT Baker. Ovalbumin peptide 323-339, (sequence: H-Ile-Ser-Gln-Ala-Val-His-Ala-Ala-His-Ala-Gludle-Asn-Glu-Ala-Gly-Arg-NH2 (SEQ ID NO: 4), acetate salt, Lot# B06395) is obtained from Bachem Americas Inc. (3132 Kashiwa Street, Torrance Calif. 90505), PLA-CO2H (100DL2A) is obtained from SurModics Pharmaceuticals (756 Tom Martin Drive, Birmingham, Ala. 35211), PLGA-R848, PLA-R848, and PLA-PEG-X conjugates are prepared as described in above examples.

The above materials are used to prepare the following solutions:

1. PLGA-R848 or PLA-R848 conjugate in methylene chloride @ 100 mg/mL

2. PLA-PEG-X in methylene chloride @ 100 mg/mL

3. PLA-CO2H in methylene chloride @ 100 mg/mL

4. Ovalbumin peptide 323-339 in 0.13N HCl @ 70 mg/mL

5. Polyvinyl alcohol in 100 mM pH 8 phosphate buffer @50 mg/mL

Solution #1 (0.50 mL), solution #2 (0.25 mL) and solution #3 (0.25 mL) are combined and solution #4 in 0.13N HCl (0.1 mL) is added in a small vessel and the mixture is sonicated at 50% amplitude for 40 seconds using a Branson Digital Sonifier 250. To this emulsion is added solution #5 (2.0 mL) and sonication at 30% amplitude for 40 seconds using the Branson Digital Sonifier 250 to the second emulsion.

This is added to a stirring beaker containing a 70 mM pH 8 phosphate buffer solution (30 mL), and this mixture is stirred at room temperature for 2 hours to form the nanocarriers.

To wash the nanocarriers, a portion of the nanoparticle dispersion (26.5 mL) is transferred to a 50 mL centrifuge tube and spun at 9500 rpm (13,800 g) for one hour at 4° C., the supernatant is removed, and the pellet is re-suspended in 26.5 mL of phosphate buffered saline. The centrifuge procedure is repeated and the pellet is re-suspended in 8.3 g of phosphate buffered saline for a final nanocarrier dispersion of about 10 mg/mL containing encapsulated ova peptide.

In a similar manner, nanocarrier without ova peptide is also prepared where solution #4 is eliminated in the preparation.

In a similar manner, nanocarrier without PLGA-R848 or PLA-R848 was also prepared where solution #1 was eliminated in the preparation.

### Example 8

### NP-L2 Conjugation via Reaction of NP surface C6-azide with c-Terminal Alkyne Modified L2 Peptide

Materials: (a) Nanocarriers (NCs) made of PLA-PEG-C6-N3 with surface C6-N3 linker groups according to Example 7a; (b) L2 peptide (SEQ ID NO: 1) modified with Lys C-terminal alkyne group of the following sequence:

(SEQ ID NO: 3)
L2 peptide Ala(15)-Thr-Gln-Leu-Tyr-Lys-Thr-Cys
(22)-Lys-Gln-Ala-Gly-Thr-Cys(28)-Pro-Pro-Asp-
Ile-Ile-Pro-Lys-Val(36)-Lys(5-hexynoyl)-NH2
(Cys(22)-Cys(28) disulfide)

Procedure: To a suspension of the NCs made by the general procedure of Example 7a (28 mg/mL in PBS (pH 7.4 buffer), 0.5 mL) was added a solution of the modified L2 peptide containing the C-terminal alkyne linker (Lot No. B06055, Bachem Biosciences, Inc, MW 2595, TFA salt, 10.4 mg in 1 mL PBS solution) with gentle mixing. A solution of CuSO4 (100 mM in H2O, 0.10 mL) and a solution of copper (I) ligand, Tris(3-hydroxypropyltriazolylmethyl)amine (THPTA) (200 mM in H2O, 0.10 mL) were mixed and the resulting blue solution was added to the NCs and L2 peptide suspension, followed by a solution sodium ascorbate (200 mM in H2O, 0.2 mL). The resulting suspension was stirred under argon at rt in dark for 1 h and then at 4 C overnight. The suspension was then diluted with PBS buffer (pH 7.4) to 5 mL and centrifuged to remove the supernatant. The residual was pellet washed with 3×5 mL PBS buffer. The washed NC-L2 peptide conjugates were then re-suspended in PBS buffer at ca. 5 mg/mL concentration and stored frozen until further analysis and biological tests.

### Example 9

### NP-L2 Conjugation via Reaction of NP Surface PEG3-azide with c-Terminal Alkyne Modified L2 Peptide

Materials: (a) Nanocarriers (NCs) made of PLA-PEG-PEG3-N3 (Example 2) with surface PEG3-N3 linker groups according to Example 7a; (b) L2 peptide (SEQ ID NO: 1) modified with Lys C-terminal alkyne group of the following sequence:

(SEQ ID NO: 3)
L2 peptide Ala(15)-Thr-Gln-Leu-Tyr-Lys-Thr-Cys
(22)-Lys-Gln-Ala-Gly-Thr-Cys(28)-Pro-Pro-Asp-
Ile-Ile-Pro-Lys-Val(36)-Lys(5-hexynoyl)-NH2
(Cys(22)-Cys(28) disulfide)

The NC-L2 conjugates were prepared in the same manner as above Example 8.

### Example 10

### NP-L2 Conjugation via Reaction of NP Surface Hydrazide with c-Terminal Ketone Modified L2 Peptide (Prophetic)

Materials: (a) NCs made of PLA-PEG-CONHNH2 (Example 7b) with surface hydrazide linker groups; (b) L2 peptide (SEQ ID NO: 1) modified with C-terminal ketone group of the following sequence:

(SEQ ID NO: 5)
L2 peptide Ala(15)-Thr-Gln-Leu-Tyr-Lys-Thr-Cys
(22)-Lys-Gln-Ala-Gly-Thr-Cys(28)-Pro-Pro-Asp-
Ile-Ile-Pro-Lys-Val(36)-Gly-NH-CH2-CH2-NH-CO-
CH2-CH2-CO-CH3 (Cys(22)-Cys(28) disulfide)

Procedure: To a suspension of the NCs made by general procedure (28 mg/mL in PBS (pH 7.4 buffer), 0.5 mL) is added a solution of the modified L2 peptide containing the C-terminal ketone linker groups (Lot No. H790, prepared by C S Bio Co., MW 2571, TFA salt, 10.4 mg in 1 mL PBS solution) with gentle mixing. The resulting suspension is stirred at 4 C overnight and then diluted with PBS buffer (pH 7.4) to 5 mL and centrifuged to remove the supernatant. The residual is pellet washed with 3×5 mL PBS buffer. The washed NC-L2 peptide conjugates are then re-suspended in PBS buffer at ca. 5 mg/mL concentration and stored frozen until further analysis and biological tests.

### Example 11

### NP-L2 Conjugation via Reaction of NP Surface Maleimide with c-Terminal Thiol Modified L2 Peptide (Prophetic)

Materials: (a) NCs made of PLA-PEG-MAL (Example 7b) with surface maleimide linker groups; (b) L2 peptide (SEQ ID NO: 1) modified with C-terminal thiol group of the following sequence:

(SEQ ID NO: 6)
L2 peptide Ala(15)-Thr-Gln-Leu-Tyr-Lys-Thr-Cys
(22)-Lys-Gln-Ala-Gly-Thr-Cys(28)-Pro-Pro-Asp-
Ile-Ile-Pro-Lys-Val(36)-Gly-NH-CH2-CH2-NH-C(=NH)-
CH2-CH2-CH2-SH (Cys(22)-Cys(28) disulfide)

Procedure: A suspension of the NCs in PBS (pH 7.4) made by general procedure is concentrated and resuspended in pH 9.0 buffer (28 mg/mL in PBS, pH 9.0, 0.5 mL) and degassed with argon. A solution of the modified L2 peptide (10 mg, in 1 mL PBS, pH 9.0 buffer) containing the C-terminal thiol linker groups is added with gentle mixing under argon. The resulting suspension is stirred at 4 C overnight in dark and then diluted with PBS buffer (pH 7.4) to 5 mL and centrifuged to remove the supernatant. The residual is pellet washed with 3×5 mL PBS buffer. The washed NC-L2 peptide conjugates are then re-suspended in PBS buffer at ca. 5 mg/mL concentration and stored frozen until further analysis and biological tests.

### Example 12

### NP-L2 Conjugation via Reaction of NP Surface Thiol with c-Terminal Activated Alkene Modified L2 Peptide (Prophetic)

Materials: (a) NCs made of PLA-PEG-SH (Example 7b) with surface thiol linker groups; (b) L2 peptide (SEQ ID NO: 1) modified with C-terminal activated alkene group of the following sequence:

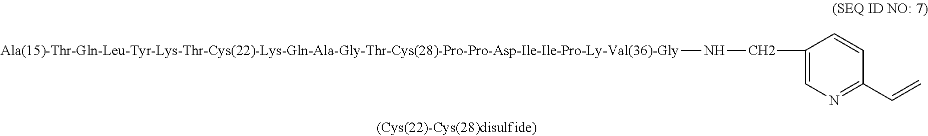

Procedure: Procedure: A suspension of the NCs in PBS (pH 7.4) made by general procedure is concentrated and re-suspended in pH 9.0 buffer (28 mg/mL in PBS, pH 9.0, 0.5 mL) and degassed with argon. A solution of the modified L2 peptide (10 mg, in 1 mL PBS, pH 9.0 buffer) containing the C-terminal activated alkene linker groups is added with gentle mixing under argon. The resulting suspension is stirred at 4 C overnight in dark and then diluted with PBS buffer (pH 7.4) to 5 mL and centrifuged to remove the supernatant. The residual is pellet washed with 3×5 mL PBS buffer. The washed NC-L2 peptide conjugates are then re-suspended in PBS buffer at ca. 5 mg/mL concentration and stored frozen until further analysis and biological tests.

### Example 13

### Mouse Immunizations with L2-Peptide Nanocarriers

In order to detect the ability of L2-coated nanocarriers to generate an antibody-driven immune response to L2 peptide (SEQ ID NO: 3), the serum anti-L2 antibody titers were measured at various timepoints after immunization with L2-peptide nanocarriers. C57BL/6 mice (5 per group) were injected subcutaneously in both hindlimbs with 30 μL/limb of 1.67 mg/mL nanoparticles for a total amount of 100 μg of nanoparticles/mouse (primary immunization) (see Table 1 below for experimental layout of groups and corresponding nanoparticle formulations). Nanocarriers were made up of 4 different formulations. The first was nanocarriers made according to Example 8; the nanocarriers contained PLGA-R848, PLA-PEG-C6-linker-L2, and ova peptide. The R848 release rate at 24 hours was 4.2 μg/mg of nanoparticles and the ova peptide load was 0.3%. The second formulation was made according to Example 8, and contained PLA-R848, PLA-PEG-C6-linker-L2, and ova peptide. The R848 release rate at 24 hours was 1.9 μg/mg of nanoparticles and the ova peptide load was 0.9%. The third formulation was made according to Example 8, and contained PLGA-R848 and PLA-PEG-C6-linker-L2 without ova peptide. The R848 release rate at 24 hours was 4.6 μg/mg of nanoparticles. The fourth formulation was made according to Example 8, and contained PLA-R848 and PLA-PEG-C6-linker-L2 without ova peptide. The R848 release rate at 24 hours was 3.4 μg/mg of nanoparticles. Control mice received either 100 μg of L2 peptide or 100 μg of L2 peptide with 100 μg of alum. At 14 and 28 days after the initial injection, mice were injected with booster shots at the same dose as the original immunization. Tail vein blood was collected at 26 and 40 days after immunization. Serum was isolated and stored at −20° C. until assayed. For groups 1 and 2, mice were given an additional boost at day 141 and serum was also isolated at days 139, 153, 156, and 415.

TABLE 1
Immunization layout for L2 peptide nanoparticles in mice.

| | | | | | R848 | R848 release | Ova |
|---|---|---|---|---|---|---|---|
| | Immunized | External | Conjugate | Polymer | Load | (24 hrs, | Peptide |
| Gr. # | with | adjuvant | Lot # | Description | (%) | μg/mg-NP) | Load (%) |
| 1 | S0833-39 | None | S0819-58 | PLGA-R848 | 2.2 | 4.2 | 0.3 |
| 2 | S0833-40 | None | S0805-88 | PLA-R848 | 2.2 | 1.9 | 0.9 |
| 3 | S0833-49 | None | S0819-58 | PLGA-R848 | 1.8 | 4.6 | None |
| 4 | S0833-50 | None | S0805-88 | PLA-R848 | 2.6 | 3.4 | None |
| 5 | L2 peptide | None | N/A | N/A | N/A | N/A | N/A |
| | (SEQ ID | | | | | | |
| | NO: 3) | | | | | | |
| 6 | L2 peptide + | Alum | N/A | N/A | N/A | N/A | N/A |
| | alum | | | | | | |
| | (SEQ ID | | | | | | |
| | NO: 3) | | | | | | |

L2 peptide ELISA: Flat bottom 96 well plates were coated with 1004 of 0.02% w/v PLA-PEG-linker-L2 and incubated overnight at 4° C. Plates were washed 3 times with wash buffer (PBS with 0.05% Tween-20) and wells were blocked for 2 hours at room temperature with 200 μL of diluent (10% fetal bovine serum in phosphate buffered saline). After blocking, plates were washed 3 times with wash buffer and serum samples were added to wells. Samples were titrated using 3-fold dilutions and incubated for 2 hours at room temperature. Plates were washed 3 times with wash buffer and incubated for one hour at room temperature with 0.5 μg/mL biotinylated goat anti-mouse Ig (BD Biosciences 553199). Plates were washed 3 times with wash buffer and streptavidin-horseradish peroxidase (BD Biosciences 554066) diluted 1:1000 was added to each well and incubated for 30 minutes at room temperature. Plates were washed 3 times (with a 30 second soak during each wash step) and tetramethylbenzidine (TMB) substrate was added to the plates. After 15 minutes of development with TMB substrate, the reaction was stopped with 2NH₂SO₄ and the optical density was read at 450 with subtraction at 570 nm. The half maximal effective concentration (EC50) of anti-L2 antibody was calculated based on the generated four-parameter logistic curve-fit graph.

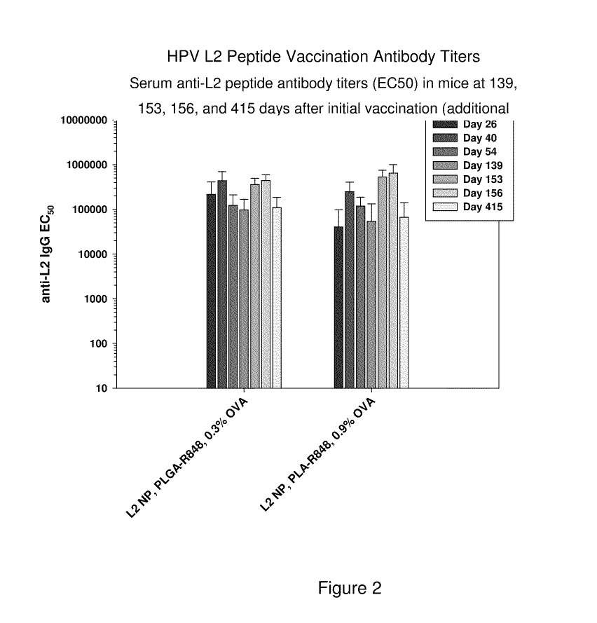

Results: Mice immunized with free L2 peptide (with or without alum) generated EC50 anti-L2 antibody titers below the level of detection for the ELISA (1:100 starting dilution for serum samples) (FIG. 1). Conjugation of L2 peptide to the nanocarriers significantly increased the anti-L2 antibody titers compared to mice immunized with L2 peptide alone (with or without alum) (FIG. 1). Mice immunized with nanocarriers containing PLGA-R848, PLA-PEG-linker-L2, and ova peptide generated EC50 anti-L2 antibody titers of 216000±195000, 439000±266000, and 123000±88000 at days 26, 40, and 54 after immunization, respectively (FIG. 1). These mice were bled at day 139 (prior to receiving an additional boost at day 141) and their titers at days 139, 153, 156, and 415 were 97000±72000, 357000±141000, 442000±157000, and 109000±77000, respectively (FIG. 2). Mice immunized with nanocarriers containing PLA-R848, PLA-PEG-linker-L2, and ova peptide generated EC50 anti-L2 antibody titers of 40400±57100, 249000±160000, and 119000±70000 at days 26, 40, and 54 after immunization, respectively. These mice were bled at day 139 (prior to receiving an additional boost at day 141) and their titers at days 139, 153, 156, and 415 were 54000±79000, 531000±224000, 647000±355000, and 66000±74000, respectively (FIG. 2). These two formulations generated long-lasting anti-L2 antibodies (over one year in mice). The lack of ova peptide in the nanocarriers drastically decreased the anti-L2 antibody titers. Mice immunized with nanocarriers containing PLGA-R848 and PLA-PEG-linker-L2 without ova peptide had EC50 anti-L2 antibody titers of 1080±1000, 23800±20400, and 11400±10500 at days 26, 40, and 54 after immunization, respectively. Mice immunized with nanocarriers containing PLA-R848 and PLA-PEG-linker-L2 without ova peptide generated EC50 anti-L2 antibody titers of 2110±1910, 3550±2710, and 1640±1520 at days 26, 40, and 54 after immunization, respectively (FIG. 1). Altogether, these data indicate that conjugation of L2 peptide to the surface of nanocarriers leads to significantly improved antibody titers after immunization. Inclusion of the ova peptide in L2 peptide nanocarriers significantly increased the antibody response. Of the two nanocarriers that contained the ova peptide that generated the best anti-L2 antibody titers, the nanocarriers with PLGA-R848 that had a faster R848 release rate (4.6 μg/mg of nanocarriers) after 24 hours (i.e. larger cumulative 24 hour release) generated higher antibody titers than the nanocarriers with PLA-R848 that had a slower (i.e. smaller cumulative 24 hour release) R848 release rate (3.4 μg/mg of nanocarriers after 24 hours), even though the PLGA-R848 nanocarriers contained less ova peptide than the PLA-R848 nanocarriers.

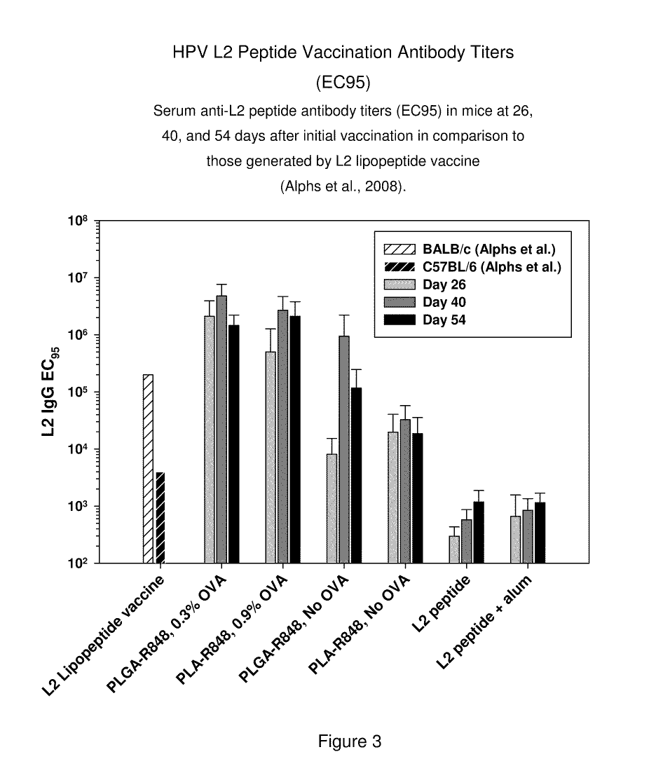

A previous study using a lipopeptide L2 vaccine reported data using endpoint titers (Alphs et al., 2008). Since our data were generated using EC50 titer values, we calculated EC95 titer values in order to compare the efficacy of our L2-peptide nanoparticle vaccine (C57BL/6 mice) to their lipopeptide vaccine in C57BL/6 and BALB/c mice (based on FIG. 2 in Alphs et al., 2008) (FIG. 3). The lipopeptide vaccine generated anti-L2 endpoint titers at day 70 of ˜200000 in BALB/c mice and ˜4000 in C57BL/6 mice. In contrast, our L2-peptide nanoparticle vaccine in C57BL/6 mice generated EC95 titers at day 54 of 1.5×10⁶±7.6×10⁵ in mice immunized with nanocarriers containing PLGA-R848, PLA-PEG-linker-L2, and ova peptide (FIG. 2). Our anti-L2 antibody titers were 375 times greater in C57BL/6 mice than those generated in C57BUc mice using a L2 lipopeptide vaccine (Alphs et al., 2008). In addition, our titers in C57BL/6 mice were 7.5 times greater than those generated in BALB/c mice using a L2 lipopeptide vaccine (Alphs et al., 2008). These data demonstrate that our L2-peptide nanoparticle vaccine generates a more robust anti-L2 peptide antibody response than previously generated L2-peptide vaccines.

### HPV Neutralization Assay:

Serum from mice immunized with nanocarriers containing PLGA-R848, PLA-PEG-linker-L2, and ova peptide or PLA-R848, PLA-PEG-linker-L2, and ova peptide were analyzed for their ability to neutralize HPV pseudovirions following a standard HPV pseudovirion neutralization assay (Buck et al., 2005). Briefly, serum from immunized mice was incubated with HPV pseudovirions from the following HPV types: 6, 16, 18, 31, 45, and 58. Serum and pseudovirions were then incubated with 293TT cells. Pseudovirions contain a reporter plasmid that encodes for secreted alkaline phosphatase (SEAP). Infected cells express high levels of SEAP that can be measured using chemiluminescence. When sufficient antibody is present to neutralized the pseudovirions, it results in a decrease of SEAP expression. The results are expressed as the titer of the reciprocal of the highest dilution of serum that reduces SEAP activity by 50% or more compared to the control (pseudovirions without serum antibodies). Additionally, serum antibody neutralization of HPV type 16 was tested using HeLa cells and pseudovirions containing a GFP reporter.

### Neutralization Assay Results:

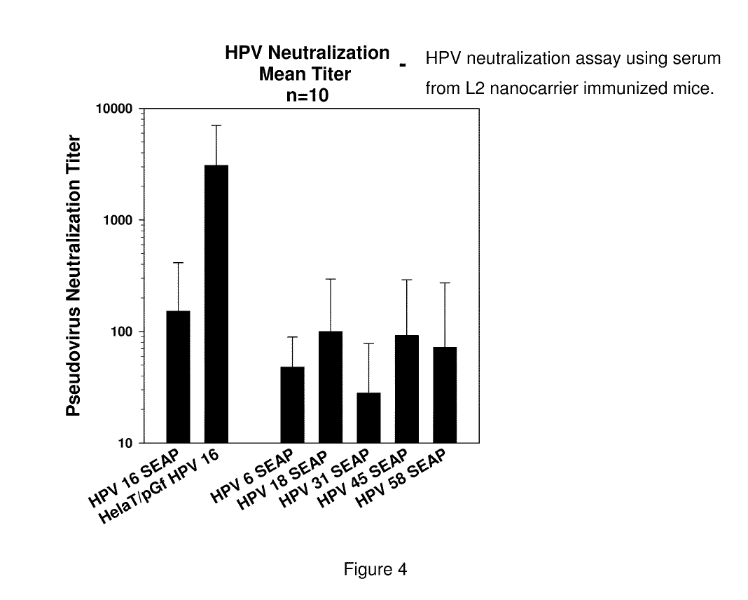

Immunization with L2 nanocarriers resulted in neutralization of HPV pseudovirions from types 16, 6, 18, 31, 45, and 58 as detected by the SEAP assay (FIG. 4). In addition, a second neutralization assay utilizing pseudovirions with a GFP reporter indicated that serum from L2 nanocarrier immunized mice neutralizes HPV type 16 pseudovirions. Together these data indicated that the L2 nanocarriers generate antibodies that recognize L2 on pseudovirions and neutralize the pseudovirions, thereby preventing infection of cells.

### Example 14

### In Vivo Testing of Synthetic Nanocarriers

To confirm positive results for generation of antibodies to the L2 peptide, nanocarrier formulations containing L2 peptide conjugated to PLA-PEG-PEG3-Azide with or without R848 were tested. The first formulation was nanocarriers made according to Example 9; the nanocarriers contained PLGA-R848, PLA-PEG-PEG3-linker-L2, and ova peptide. The R848 release rate at 24 hours was 18 μg/mg of nanoparticles and the ova peptide load was 1.5%. The L2 peptide load was 2.9%. The second formulation was nanocarriers made according to Example 9; the nanocarriers contained PLA-PEG-PEG3-linker-L2 and ova peptide without PLGA-R848. The ova peptide load was 2.4% and the L2 peptide load was 3.2%. The third formulation was nanocarriers made according to Example 9; the nanocarriers contained PLA-PEG-PEG3-linker-L2 and ova peptide without PLGA-R848 but the nanoparticles were mixed with 100 μg alum as adjuvant. The ova peptide load was 2.4% and the L2 peptide load was 3.2%. C57BL/6 mice (5 per group) were injected subcutaneously in both hindlimbs at days 0, 13, and 32 with 30 μL/limb of 1.67 mg/mL nanoparticles for a total amount of 100 μg of nanoparticles/mouse (primary immunization) (see Table 2 below for experimental layout of groups and corresponding nanoparticle formulations). Serum was collected at days 12, 24, 45, and 59 and analyzed by ELISA for antibodies to L2 peptide as described above (see “L2 peptide ELISA” section).

TABLE 2
Immunization layout for L2 peptide nanoparticles in mice.

| | | | | | R848 | |
|---|---|---|---|---|---|---|
| | | | | | release | Ova |
| | | | | R848 | (24 hrs, | Peptide |
| | | | L2 | Load | μg/mg- | Load |
| Gr. # | Immunized with | NP Lot # | Load | (%) | NP) | (%) |
| 1 | NP-L2 (+R848) | S0856-93 | 2.9 | 3.0 | 18 | 1.5 |
| 2 | NP-L2 (no R848) | S0856-94 | 3.2 | N/A | N/A | 2.4 |
| 3 | NP-L2 (no R848) + | S0856-94 | 3.2 | N/A | N/A | 2.4 |
| | alum | | | | | |

### Results:

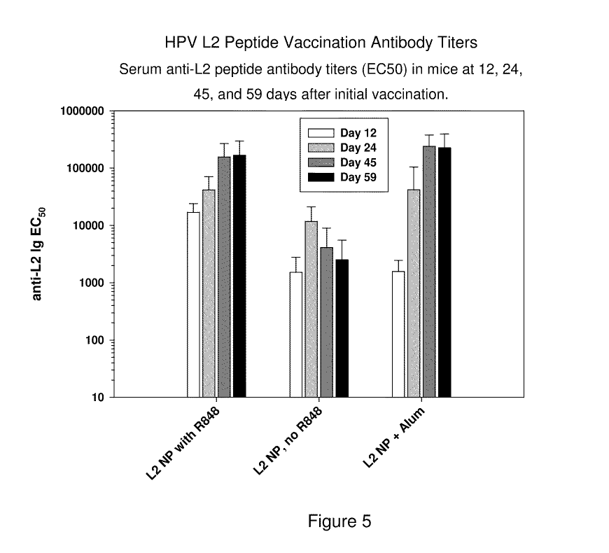

The addition of R848 in nanocarriers led to an increase of antibody titers to L2 peptide that was similar to that seen with L2 nanocarriers admixed with alum. Both formulations led to higher titers than L2 nanocarriers that lacked adjuvant. Mice immunized with nanocarriers containing PLGA-R848, PLA-PEG-PEG3-linker-L2, and ova peptide generated EC50 anti-L2 antibody titers of 17000±7000, 41000±29000, 156000±113000, and 168000±129000 at days 12, 24, 45, and 59 after immunization, respectively (FIG. 5). The lack of adjuvant in the nanocarriers drastically decreased anti-L2 antibody titers. Mice immunized with nanocarriers containing PLA-PEG-PEG3-linker-L2 and ova peptide (no adjuvant, such as R848) generated EC50 anti-L2 antibody titers of 1500±1300, 12000±9300, 4100±4800, and 2500±3000 at days 12, 24, 45, and 59 after immunization, respectively. The addition of alum as an adjuvant to the nanocarriers that did not contain adjuvant within the nanocarrier increased the anti-L2 antibody titers. Mice immunized with nanocarriers containing PLA-PEG-PEG3-linker-L2 and ova peptide (no adjuvant, such as R848) and admixed with alum generated EC50 anti-L2 antibody titers of 1600±870, 42000±63000, 240000±137000, and 226000±168,000 at days 12, 24, 45, and 59 after immunization, respectively. Together, these data show that L2 nanocarriers injected in the presence of adjuvant generate high titers of anti-L2 antibodies and the absence of adjuvant decreases the antibody response.

### Example 15

### In Vivo Testing of Synthetic Nanocarriers

L2 was attached to nanocarriers using two different polymer formulations (PLA-PEG-C6-N3 or PLA-PEG-PEG3-N3) to determine whether the PEG composition affected antibody generation to L2 peptide. The first formulation was nanocarriers made according to Example 8; the nanocarriers contained PLGA-R848, PLA-PEG-C6-linker-L2, and ova peptide. The R848 load was 2.9% and the ova peptide load was 0.9%. The second formulation was nanocarriers made according to Example 9; the nanocarriers contained PLGA-R848, PLA-PEG-PEG3-linker-L2, and ova peptide. The R848 load was 4.0% and the ova peptide load was 2.4%. C57BL/6 mice (5 per group) were injected subcutaneously in both hindlimbs at days 0, 14, and 28 with 30 μL/limb of 1.67 mg/mL nanoparticles for a total amount of 100 μg of nanoparticles/mouse (primary immunization) (see Table 3 below for experimental layout of groups and corresponding nanoparticle formulations). Serum was collected at days 27, 40, and 54 and analyzed by ELISA for antibodies to L2 peptide as described above (see “L2 peptide ELISA” section).

TABLE 3
Immunization layout for L2 peptide nanoparticles in mice.

| | | | Ova | R848 | |
|---|---|---|---|---|---|
| | | | Peptide | (% | |
| Gr. # | Nanoparticle Description | NP Lot # | (% w/w) | w/w) | Formulation notes |
| 1 | NP-L2 via PLA-PEG-C6-N3 | S888-49A | 0.9 | 2.9 | 25% PLA-PEG-C6-N3; 50% |
| | | | | | PLGA-R848; 25% 100DL2A |
| 2 | NP-L2 via PLA-PEG-PEG3-N3 | S888-49B | 2.4 | 4.0 | 25% PLA-PEG-PEG3-N3; 50% |
| | | | | | PLGA-R848; 25% 100 DL2A |

### Results:

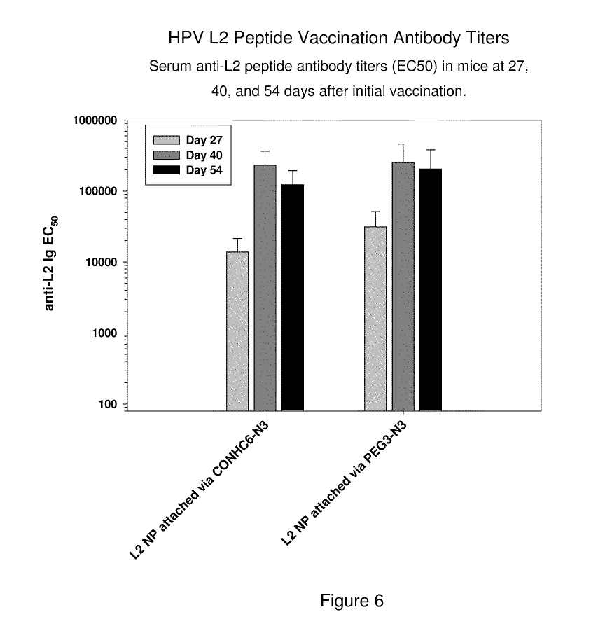

The two formulations tested generated comparable titers. Both nanoparticles generated robust anti-L2 peptide antibody titers by day 40 after immunization. Mice immunized with nanocarriers containing PLGA-R848, PLA-PEG-C6-linker-L2, and ova peptide generated EC50 anti-L2 antibody titers of 14000±7500, 232000±133000, and 123000±71000 at days 27, 40, and 54 after immunization, respectively (FIG. 6). Mice immunized with nanocarriers containing PLGA-R848, PLA-PEG-PEG3-linker-L2, and ova peptide generated EC50 anti-L2 antibody titers of 31000±20000, 252000±210000, and 205000±177000 at days 27, 40, and 54 after immunization, respectively (FIG. 6).

### Citation for Neutralization Assay:

Buck C B, Pastrana D V, Lowy D R, Schiller J T. Generation of HPV pseudovirions using transfection and their use in neutralization assays. Methods Mol. Med. 2005; 119:445-62.

TABLE 4
Antigenic Sequences

| Protein | | | | |
|---|---|---|---|---|
| (L1 or | HPV | Amino acid | | |
| L2) | Type(s) | residues | Peptide sequence | Source |
| L1 | 16 | 267-281 | VGENVPDDLYIKGSG | Cason, J., D. Patel, J. Naylor, D. Lunney, P. |
| | | | (SEQ. ID NO: 8) | S. Shepherd, J. M. Best, and D. J. McCance. 1989. |
| | | | | Identification of immunogenic regions of the |
| | | | | major coat protein of human papillomavirus |
| | | | | type 16 that contain type-restricted epitopes. J. |
| | | | | Gen. Virol. 70: 2973-2987. |
| L1 | 16 | 473-492 | GLKAKPKFTLGKRKAT | Cason, J., P. K. Kambo, J. M. Best, and |
| | | | PTT (SEQ. ID NO: | D. J. McCance. 1992. Detection of antibodies to a |
| | | | 9) | linear epitope on the major coat protein (L1) |
| | | | | of human papillomavirus 16 (HPV16) in sera from |
| | | | | patients with cervical intraepithelial neoplasia |
| | | | | and in children. Int. J. Cancer 50: 349-355. |
| L1 | 16 | 268-281 | ENVPDDLYIKGSGS | Touzé, A., C. Dupuy, D. Mahé, P.-Y. Sizaret, and |
| | | | (SEQ. ID NO: 10) | P. Coursaget. 1998. Production of recombinant virus-like |
| | | | | particles from human papillomavirus type 6 and 11, |
| | | | | and study of serological reactivities between |
| | | | | HPV 6,11,16 and 45 by ELISA: implications for |
| | | | | papillomavirus prevention and detection. FEMS Microbiol. |
| | | | | Lett. 160: 111-118. |
| L1 | 16 | 111-130 | QPLGVGISGHPLLNKL | Christensen, N. D., J. Dillner, C. Eklund, J. J. Carter, |
| | | | DDTE (SEQ. ID | G. C. Wipf, C. A. Reed, N. M. Cladel, and D. A. Galloway. |
| | | | NO: 11) | 1996. Surface conformational and linear epitopes on |
| | | | | HPV-16 and HPV-18 L1 virus-like particles as defined by |
| | | | | monoclonal antibodies. Virology 223: 174-184. |
| L1 | 16 | 261-280 | FNRAGTVGENVPDDL | Christensen, N. D., J. Dillner, C. Eklund, J. J. Carter, |
| | | | YIKGS (SEQ ID | G. C. Wipf, C. A. Reed, N. M. Cladel, and D. A. Galloway. |
| | | | NO: 12) | 1996. Surface conformational and linear epitopes on |
| | | | | HPV-16 and HPV-18 L1 virus-like particles as defined by |
| | | | | monoclonal antibodies. Virology 223: 174-184. |
| L1 | 16 | 396-415 | STILEDWNFGLQPPPG | Christensen, N. D., J. Dillner, C. Eklund, J. J. Carter, |
| | | | GTLE (SEQ ID NO: | G. C. Wipf, C. A. Reed, N. M. Cladel, and D. A. Galloway. |
| | | | 13) | 1996. Surface conformational and linear epitopes on |
| | | | | HPV-16 and HPV-18 L1 virus-like particles as defined by |
| | | | | monoclonal antibodies. Virology 223: 174-184. |
| L1 | 16 | 174-185 | PCTNVAVNPGDC | Christensen, N. D., J. Dillner, C. Eklund, J. J. Carter, |
| | | | (SEQ ID NO: 14) | G. C. Wipf, C. A. Reed, N. M. Cladel, and D. A. Galloway. |
| | | | | 1996. Surface conformational and linear epitopes on |
| | | | | HPV-16 and HPV-18 L1 virus-like particles as defined by |
| | | | | monoclonal antibodies. Virology 223: 174-184. |
| L1 | 16 | -10-10 | DVNVYHIFFQMSLWL | Christensen, N. D., J. Diliner, C. Eklund, J. J. Carter, |
| | | | PSEAT (SEQ ID | G. C. Wipf, C. A. Reed, N. M. Cladel, and D. A. |
| | | | NO: 15) | Galloway. 1996. Surface conformational and linear |
| | | | | epitopes on HPV-16 and HPV-18 L1 virus-like particles as |
| | | | | defined by monoclonal antibodies. Virology 223: 174-184. |
| L1 | 18 | 297-416 | SSILEDWNFGVPPPPT | Christensen, N. D., J. Diliner, C. Eklund, J. J. Carter, |
| | | | TSLV (SEQ ID NO: | G. C. Wipf, C. A. Reed, N. M. Cladel, and D. A. Galloway. |
| | | | 16) | 1996. Surface conformational and linear epitopes on |
| | | | | HPV-16 and HPV-18 L1 virus-like particles as defined |
| | | | | by monoclonal antibodies. Virology 223: 174-184. |
| L1 | 11 | 120-140 | LNKYDDVENSGGYGG | Ludmerer, S.W., Benincasa, D., Mark 3rd, G.E., |
| | | | NPGQDN (SEQ ID | Christensen, N.D., 1997. A neutralizing epitope |
| | | | NO: 17) | of human papillomavirus type 11 is principally |
| | | | | described by a continuous set of residues |
| | | | | which overlap a distinct linear, surface-exposed |
| | | | | epitope. J. Virol. 71, 834-3839 |
| L1 | 6 | 49-54 | FSIKRA(SEQ ID | McClements, W.L., Wang, X-M., Ling, J.C., Skulsky, |
| | | | NO: 18) | D.M., Christensen, N.D., Jansen, K.U., Ludmerer, |
| | | | | S.W., 2001. A novel human papillomavirus type 6 |
| | | | | neutralizing domain comprising two discrete regions of |
| | | | | the major capsid protein L1. Virology 289, 262-268. |
| L1 | 6 | 169-178 | KQCTNTPVQA (SEQ | McClements, W.L., Wang, X-M., Ling, J.C., Skulsky, |
| | | | ID NO: 19) | D.M., Christensen, N.D., Jansen, K.U., Ludmerer, |
| | | | | S.W., 2001. A novel human papillomavirus type 6 |
| | | | | neutralizing domain comprising two discrete regions of |
| | | | | the major capsid protein L1. Virology 289, 262-268. |
| L1 | 16 | 55-74 | PNNNKILVPKVSGLQY | Urquiza M, Guevara T, Espejo F, Bravo MM, Rivera Z, |
| | | | RVFR (SEQ. ID | Patarroyo ME. Two L1-peptides are excellent tools for |
| | | | NO: 20) | serological detection of HPV-associated cervical |
| | | | | carcinoma lesions. Biochem Biophys Res Commun |
| | | | | 2005; 332(1): 224-32. |
| L1 | 16 | 275-294 | LYIKGSGSTANLASSN | Urquiza M, Guevara T, Espejo F, Bravo MM, Rivera Z, |
| | | | YFPT (SEQ. ID | Patarroyo ME. Two L1-peptides are excellent tools for |
| | | | NO: 21) | serological detection of HPV-associated cervical |
| | | | | carcinoma lesions. Biochem Biophys Res Commun |
| | | | | 2005; 332(1): 224-32. |
| L1 | 16 | 416-436 | EDTYRFVTQAIACQKH | Urquiza M, Guevara T, Sanchez R, Vanegas M, Patarroyo |
| | | | TPPA (SEQ. ID | ME. A non-variable L1-peptide displays high sensitivity |
| | | | NO: 22) | and specificity for detecting women having human |
| | | | | papillomavirus-associated cervical lesions. Peptides. |
| | | | | 2008 June; 29(6): 957-62. |
| L1 | 31 | 267-285 | TVGESVPTDLYIKGSG | Fleury MJ, Touzé A, Alvarez E, Carpentier G, Clavel |
| | | | STA (SEQ. ID NO: | C, Vautherot JF, Coursaget P. Identification |
| | | | 23) | of type-specific and cross-reactive |
| | | | | neutralizing conformational epitopes on the |
| | | | | major capsid protein of human papillomavirus |
| | | | | type 31. Arch Virol. 2006 August; 151(8): 1511-23. |
| L1 | 16 | 264-283 | AGTVGENVPDDLYIKG | Reddy KJ, Banapour B, Anderson DE, Lee SH, Marquez JP, |
| | | | SGST (SEQ. ID | Carlos MP, Torres JV. Induction of immune responses |
| | | | NO: 24) | against human papillomaviruses by hypervariable |
| | | | | epitope constructs. Immunology. 2004 June; 112(2): 321-7. |
| L1 | 18 | 264-283 | AGTMGDTVPQSLYIKG | Reddy KJ, Banapour B, Anderson DE, Lee SH, Marquez JP, |
| | | | TGMR (SEQ. ID | Carlos MP, Torres JV. Induction of immune responses |
| | | | NO: 25) | against human papillomaviruses by hypervariable |
| | | | | epitope constructs. Immunology. 2004 June; 112(2): 321-7. |
| L1 | 31 | 264-283 | SGTVGESVPTDLYIKG | Reddy KJ, Banapour B, Anderson DE, Lee SH, Marquez JP, |
| | | | SGST (SEQ. ID | Carlos MP, Torres JV. Induction of immune responses |
| | | | NO: 26) | against human papillomaviruses by hypervariable |
| | | | | epitope constructs. Immunology. 2004 June; 112(2): 321-7. |
| L1 | 45 | 264-283 | AGVMGDTVPTDLYIKG | Reddy KJ, Banapour B, Anderson DE, Lee SH, Marquez JP, |
| | | | TSANMR (SEQ. ID | Carlos MP, Torres JV. Induction of immune responses |
| | | | NO: 27) | against human papillomaviruses by hypervariable |
| | | | | epitope constructs. Immunology. 2004 June; 112(2): 321-7. |
| L1 | 16 | 469-493 | LLQAGLKAKPKFTLGK | Reddy KJ, Banapour B, Anderson DE, Lee SH, Marquez JP, |
| | | | RKATPTTSS | Carlos MP, Torres JV. Induction of immune responses |
| | | | (SEQ ID NO: 28) | against human papillomaviruses by hypervariable |
| | | | | epitope constructs. Immunology. 2004 June; 112(2): 321-7. |
| L1 | 18 | 469-493 | LVQAGLRRKPTIGPRK | Reddy KJ, Banapour B, Anderson DE, Lee SH, Marquez JP, |
| | | | RSAPSATTS | Carlos MP, Torres JV. Induction of immune responses |
| | | | (SEQ. ID NO: 29) | against human papillomaviruses by hypervariable |
| | | | | epitope constructs. Immunology. 2004 June; 112(2): 321-7. |
| L1 | 31 | 469-493 | LLQAGYRARPKFKAGK | Reddy KJ, Banapour B, Anderson DE, Lee SH, Marquez JP, |
| | | | RSAPSASTT (SEQ. | Carlos MP, Torres JV. Induction of immune responses |
| | | | ID NO: 30) | against human papillomaviruses by hypervariable |
| | | | | epitope constructs. Immunology. 2004 June; 112(2): 321-7. |
| L1 | 45 | 469-493 | LVQAGLRRRPTIGPRK | Reddy KJ, Banapour B, Anderson DE, Lee SH, Marquez JP, |
| | | | RPAASTST (SEQ. | Carlos AMP, Torres JV. Induction of immune responses |
| | | | ID NO: 31) | against human papillomaviruses by hypervariable |
| | | | | epitope constructs. Immunology. 2004 June; 112(2): 321-7. |
| L2 | 6 and | 108-120 | LVEETSFIDAGAP | Kawana, K., K. Matsumoto, H. Yoshikawa, Y. Taketani, |
| | 16 | | (SEQ. ID NO: 32) | T. Kawana, K. Yoshiike, and T. Kanda. 1998. A surface |
| | | | | immunodeterminant of human papillomavirus type 16 minor |
| | | | | capsid protein L2. Virology 245: 353-359. |
| L2 | 16 | 75-112 | LGTRPPTATDTLAPVR | Schellenbacher C., Roden R., Kirnbauer R. 2009. |
| | | | PPLTVDPVGPSDPSIV | Chimeric L1-L2 virus-lik particles as potential |
| | | | SLVEET (SEQ. ID | broad-spectrum human papillomavirus vaccines. |
| | | | NO: 33) | Journal of Virology. 83(19): 10085-10095. |
| L2 | 16 | 115-154 | IDAGAPTSVPSIPPDV | Schellenbacher C., Roden R., Kirnbauer R. 2009. |
| | | | SGFSITTSTDTTPAIL | Chimeric L1-L2 virus-lik particles as potential |
| | | | DINNTVTT (SEQ. | broad-spectrum human papillomavirus vaccines. |
| | | | ID NO: 34) | Journal of Virology. 83(19): 10085-10095. |
| L2 | 16 | 149-175 | NNTVTTVTTHNNPTFT | Schellenbacher C., Roden R., Kirnbauer R. 2009. |
| | | | DPSVLQPPTPA | Chimeric L1-L2 virus-lik particles as potential |
| | | | (SEQ. ID NO: 35) | broad-spectrum human papillomavirus vaccines. |
| | | | | Journal of Virology. 83(19): 10085-10095. |
| L2 | 16 | 172-200 | PTPAETGGHFTLSSST | Schellenbacher C., Roden R., Kirnbauer R. 2009. |
| | | | ISTHNYEEIPMDT | Chimeric L1-L2 virus-lik particles as potential |
| | | | (SEQ. ID NO: 36 | broad-spectrum human papillomavirus vaccines. |
| | | | | Journal of Virology. 83(19): 10085-10095. |
| L2 | 16 | 18-38 | LYKTCKQAGTCPPDII | Kondo K., Ishii Y., Ochi H., Matsumoto T., Yoshikawa H., |
| | | | PKVEG (SEQ ID | Kanda T. Neutralization of HPV16, 18, 31, and 58 |
| | | | NO: 37) | pseudovirions with antisera induced by immunizing |
| | | | | rabbits with synthetic peptides representing segments |
| | | | | of the HPV 16 minor capsid protein L2 surface region. |
| | | | | Virology 2007; 358: 266-72. |
| L2 | 16 | 56-75 | CGGLGIGTGSGTGGRT | Kondo K., Ishii Y., Ochi H., Matsumoto T., Yoshikawa H., |
| | | | GYIPL (SEQ ID | Kanda T. Neutralization of HPV16, 18, 31, and 58 |
| | | | NO: 38) | pseudovirions with antisera induced by immunizing |
| | | | | rabbits with synthetic peptides representing segments |
| | | | | of the HPV 16 minor capsid protein L2 surface region. |
| | | | | Virology 2007; 358: 266-72. |
| L2 | 16 | 61-75 | CGTGSGTGGRTGYIPL | Kondo K., Ishii Y., Ochi H., Matsumoto T., Yoshikawa H., |
| | | | (SEQ ID NO: 39) | Kanda T. Neutralization of HPV16, 18, 31, and 58 |
| | | | | pseudovirions with antisera induced by immunizing |
| | | | | rabbits with synthetic peptides representing segments |
| | | | | of the HPV 16 minor capsid protein L2 surface region. |
| | | | | Virology 2007; 358: 266-72. |
| L2 | 16 | 64-81 | CSGTGGRTGYIPLGTR | Kondo K., Ishii Y., Ochi H., Matsumoto T., Yoshikawa H., |
| | | | PPT (SEQ ID NO: | Kanda T. Neutralization of HPV16, 18, 31, and 58 |
| | | | 40) | pseudovirions with antisera induced by immunizing |
| | | | | rabbits with synthetic peptides representing segments |
| | | | | of the HPV 16 minor capsid protein L2 surface region. |
| | | | | Virology 2007; 358: 266-72. |
| L2 | 16 | 96-115 | CDPVGPSDPSIVSLVE | Kondo K., Ishii Y., Ochi H., Matsumoto T., Yoshikawa H., |
| | | | ETSFI (SEQ ID | Kanda T. Neutralization of HPV16, 18, 31, and 58 |
| | | | NO: 41) | pseudovirions with antisera induced by immunizing |
| | | | | rabbits with synthetic peptides representing segments |
| | | | | of the HPV 16 minor capsid protein L2 surface region. |
| | | | | Virology 2007; 358: 266-72. |
| L2 | 16 | 69-81 | RTGYIPLGTRPPT | Slupetzky K, Gambhira R, Culp TD, Shafti-Keramat S, |
| | | | (SEQ ID NO: 42) | Schellenbacher C, Christensen ND, Roden RB, |
| | | | | Kirnbauer R. A papillomavirus-like particle |
| | | | | (VLP) vaccine displaying HPV16 L2 epitopes induces |
| | | | | cross-neutralizing antibodies to HPV11. Vaccine. |
| | | | | 2007 Mar. 1; 25(11): 2001-10. |
| L2 | 16 | 391-402 | SGYIPANTTIPF | Lehtinen M, Niemelä J, Dillner J, Parkkonen P, Nummi T, |
| | | | (SEQ ID NO: 43) | Liski E, Nieminen P, Reunala T, Paavonen J. Evaluation |
| | | | | of serum antibody response to a newly identified |
| | | | | B-cell epitope in the minor nucleocapsid protein L2 |
| | | | | of human papillomavirus type 16. Clin |
| | | | | Diagn Virol. 1993 August; 1(3): 153-65. |
| L2 | 16 | 197-216 | PMDTFIVSTNPNTVTS | Wikström A, Eklund C, Von Krogh G, Lidbrink P, Diliner |
| | | | STPI (SEQ ID | J. Levels of immunoglobulin G antibodies against |
| | | | NO: 44) | defined epitopes of the L1 and L2 capsid proteins |
| | | | | of human papillomavirus type 6 are elevated in |
| | | | | men with a history of condylomata acuminata. J Clin |
| | | | | Microbiol. 1992 July; 30(7): 1795-800. |
| L2 | 16 | 20-38 | KTCKQAGTCPPDIIPK | Rubio I, Bolchi A, Moretto N, Canali E, Gissmann L, |
| | | | VEG (SEQ ID NO: | Tommasino M, Müller M, Ottonello S. Potent anti-HPV |
| | | | 45) | immune responses induced by tandem repeats of the HPV16 |
| | | | | L2 (20--38) peptide displayed on bacterial thioredoxin. |
| | | | | Vaccine. 2009 Mar. 18; 27(13): 1949-56. |
| L2 | 16 | 32-51 | IIPKVEGKTIAEQILQ | Heino P, Skyldberg B, Lehtinen M, Rantala I, Hagmar |
| | | | YGSM (SEQ ID | B, Kreider JW, Kirnbauer R, Dillner J. Human |
| | | | NO: 46) | papillomavirus type 16 capsids expose multiple |
| | | | | type-restricted and type-common antigenic |
| | | | | epitopes. J Gen Virol. 1995 May; 76 (Pt 5): 1141-53. |
| L2 | 16 | 62-81 | TGSGTGGRTGYIPLGT | Heino P, Skyldberg B, Lehtinen M, Rantala I, Hagmar |
| | | | RPPT (SEQ ID | B, Kreider JW, Kirnbauer R, Dillner J. Human |
| | | | NO: 47) | papillomavirus type 16 capsids expose multiple |
| | | | | type-restricted and type-common antigenic |
| | | | | epitopes. J Gen Virol. 1995 May; 76 (Pt 5): 1141-53. |
| L2 | 16 | 212-231 | SSTPIPGSRPVARLGL | Heino P, Skyldberg B, Lehtinen M, Rantala I, Hagmar |
| | | | YSRT (SEQ. ID | B, Kreider JW, Kirnbauer R, Dillner J. Human |
| | | | NO: 48) | papillomavirus type 16 capsids expose multiple |
| | | | | type-restricted and type-common antigenic |
| | | | | epitopes. J Gen Virol. 1995 May; 76 (Pt 5): 1141-53. |
| L2 | 16 | 279-291 | DNSINIAPDPDFLDIV | Heino P, Skyldberg B, Lehtinen M, Rantala I, Hagmar |
| | | | ALHR (SEQ ID NO: | B, Kreider JW, Kirnbauer R, Dillner J. Human |
| | | | 49) | papillomavirus type 16 capsids expose multiple |
| | | | | type-restricted and type-common antigenic |
| | | | | epitopes. J Gen Virol. 1995 May; 76 (Pt 5): 1141-53. |
| L2 | 16 | 362-381 | NNGLYDIYADDFITDT | Heino P, Skyldberg B, Lehtinen M, Rantala I, Hagmar |
| | | | STTP (SEQ ID NO: | B, Kreider JW, Kirnbauer R, Dillner J. Human |
| | | | 50) | papillomavirus type 16 capsids expose multiple |
| | | | | type-restricted and type-common antigenic |
| | | | | epitopes. J Gen Virol. 1995 May; 76 (Pt 5): 1141-53. |
| L2 | 16 | 14-40 | SATQLYKTCKQAGTCP | Caldeira Jdo C, Medford A, Kines RC, Lino CA, |
| | | | PDIIPKVEGKT (SEQ | Schiller JT, Chackerian B, Peabody DS. Immunogenic |
| | | | ID NO: 51) | display of diverse peptides, including a broadly |
| | | | | cross-type neutralizing human papillomavirus L2 |
| | | | | epitope, on virus-like particles of the RNA |
| | | | | bacteriophage PP7. Vaccine. 2010 Jun. 17; 28(27): 4384-93. |
| L2 | 16 | 17-31 | QLYKTCKQAGTCPPD | Caldeira Jdo C, Medford A, Kines RC, Lino CA, |
| | | | (SEQ ID NO: 52) | Schiller JT, Chackerian B, Peabody DS. Immunogenic |
| | | | | display of diverse peptides, including a broadly |
| | | | | cross-type neutralizing human papillomavirus L2 |
| | | | | epitope, on virus-like particles of the RNA |
| | | | | bacteriophage PP7. Vaccine. 2010 Jun. 17; 28(27): 4384-93. |
| L2 | 16 | 65-81 | GTGGRTGYIPLGTRPP | Varsani A, Williamson AL, de Villiers D, |
| | | | T (SEQ ID NO: | Becker I, Christensen ND, Rybicki EP. |
| | | | 53) | Chimeric human papillomavirus type 16 (HPV-16) |
| | | | | L1 particles presenting the common neutralizing |
| | | | | epitope for the L2 minor capsid protein of |
| | | | | HPV-6 and HPV-16. J Virol. 2003 August; 77(15): 8386-93. |

1. A composition comprising:
a synthetic nanocarrier;
at least one peptide obtained from Human papillomavirus L1 or L2 capsid protein;
wherein the at least one peptide is coupled to an external surface of the synthetic nanocarrier; and
wherein if the at least one peptide obtained from Human papillomavirus L1 or L2 capsid protein comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein.

2. The composition of claim 1, wherein the at least one peptide is obtained from Human papillomavirus L1 capsid protein.

3. The composition of claim 2, wherein the peptide comprises a sequence obtained from L1 capsid protein BC loop (aa50-69), DE loop (aa110-153), EF loop (aa160-189), FG loop (aa262-291), or HI loop (aa348-360); or HPV L1 residues 1-173, 111-130, 268-281 or 427-445.

4. The composition of claim 2, wherein the peptide was obtained from Human papillomavirus type 16, 18, 26, 31, 33, 35, 39, 45, 51, 52, 53, 56, 58, 59, 66, 68, 73, or 82.

5. The composition of claim 4, wherein the peptide was obtained from Human papillomavirus type 16 or 18.

6. The composition of claim 5, wherein the peptide was obtained from Human papillomavirus type 16.

7. The composition of claim 2, wherein the peptide is covalently coupled to the external surface.

8. The composition of claim 7, wherein the peptide is covalently coupled to the external surface via a 1,2,3-triazole linker, an amide linker, a thioether linker, a disulfide linker, an amine linker, a hydrazone linker, an urea or thiourea linker, an oxime or imine linker, or a sulfonamide linker.

9. The composition of claim 2, wherein the synthetic nanocarrier comprises an adjuvant.

10. The composition of claim 2, wherein the synthetic nanocarrier comprises a CD4+ T-cell antigen.

11. The composition of claim 10, wherein the CD4+ T-cell antigen comprises a sequence obtained from, or derived from, tetanus toxoid, Epstein-Barr virus, influenza virus, respiratory syncytial virus, cytomegalovirus, adenovirus, diphtheria toxoid, PADRE peptide, α-galactosylceramide, α-linked glycosphingolipids, galactosyl diacylglycerols, lypophosphoglycan, or phosphatidylinositol tetramannoside.

12. The composition of claim 2, further comprising a pharmaceutically acceptable excipient that comprises preservative, buffer, saline, or phosphate buffered saline.

13. The composition of claim 2, wherein the synthetic nanocarrier comprises a polymeric nanoparticle, a virus-like particle, a liposome, a metallic nanoparticle, or a nanocarrier with an aspect ratio greater than 1:1.

14. The composition of claim 2, wherein more than one peptide obtained from Human papillomavirus L1 capsid protein is coupled to an external surface of the synthetic nanocarrier.

15-32. (canceled)

33. A compound comprising:
[SEQ ID NO. 1]
H-Ala-Thr-Gln-Leu-Tyr-Lys-Thr-Cys-Lys-Gln-Ala-
Gly-Thr-Cys-Pro-Pro-Asp-Ile-Ile-Pro-Lys-Val-X;
wherein X is a linker group comprising a terminal alkyne function or an azido function.

34-35. (canceled)

36. A composition comprising:
a synthetic nanocarrier;
at least one peptide obtained from Human papillomavirus L2 capsid protein, wherein the peptide comprises amino acid residues Cys22 and Cys28 of Human papillomavirus L2 capsid protein;
wherein the peptide is coupled to an external surface of the synthetic nanocarrier; and
wherein if the at least one peptide obtained from Human papillomavirus L2 capsid protein comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein.

37-51. (canceled)

52. A composition comprising:
a synthetic nanocarrier;
at least one peptide obtained from Human papillomavirus L1 capsid protein, wherein the peptide comprises a sequence obtained from L1 capsid protein BC loop (aa50-69), DE loop (aa110-153), EF loop (aa160-189), FG loop (aa262-291), or HI loop (aa348-360); or HPV L1 residues 1-173, 111-130, 268-281 or 427-445; and
wherein the peptide is coupled to an external surface of the synthetic nanocarrier.

53-63. (canceled)

64. A composition comprising:
a synthetic nanocarrier;
a universal T-cell antigen an adjuvant;
at least one peptide obtained from Human papillomavirus L2 capsid protein;
wherein the at least one peptide is coupled to an external surface of the synthetic nanocarrier;
wherein the universal T-cell antigen is coupled to the synthetic nanocarrier;
wherein the adjuvant is coupled to the synthetic nanocarrier; and
wherein if the at least one peptide obtained from Human papillomavirus L2 capsid protein comprises a peptide that comprises amino acid residues 15-36 of Human papillomavirus L2 capsid protein, then the peptide that comprises amino acid residues 15-36 of Human papillomavirus L2 capsid protein is coupled to the nanocarrier only by the C-terminal end of the peptide that comprises amino acid residues 15-36 of Human papillomavirus L2 capsid protein.

65-73. (canceled)

74. A composition comprising:
a synthetic nanocarrier;
a universal T-cell antigen;
an adjuvant;
at least one peptide obtained from Human papillomavirus L1 capsid protein;
wherein the at least one peptide is coupled to an external surface of the synthetic nanocarrier;
wherein the universal T-cell antigen is coupled to the synthetic nanocarrier; and
wherein the adjuvant is coupled to the synthetic nanocarrier.

75-84. (canceled)

85. A method comprising:
administering any one of the compositions of claim 1 to a subject.


SEQUENCE LISTING (53 sequences)

SEQ ID NO: 1 - protein, 22 aa, Artificial Sequence
  Synthetic polypeptide
ATQLYKTCKQAGTCPPDIIPKV

SEQ ID NO: 2 - protein, 22 aa, Artificial Sequence
  Synthetic polypeptide
ATQLYKTCKQAGTCPPDIIPKV

SEQ ID NO: 3 - protein, 23 aa, Artificial Sequence
  Synthetic polypeptide
ATQLYKTCKQAGTCPPDIIPKVK

SEQ ID NO: 4 - protein, 17 aa, Artificial Sequence
  Synthetic polypeptide
ISQAVHAAHAEINEAGR

SEQ ID NO: 5 - protein, 18 aa, Artificial Sequence
  Synthetic polypeptide
KTCKQAGTCPPDIIPKVG

SEQ ID NO: 6 - protein, 23 aa, Artificial Sequence
  Synthetic polypeptide
ATQLYKTCKQAGTCPPDIIPKVG

SEQ ID NO: 7 - protein, 23 aa, Artificial Sequence
  Synthetic polypeptide
ATQLYKTCKQAGTCPPDIIPKVG

SEQ ID NO: 8 - protein, 15 aa, Artificial Sequence
  Synthetic polypeptide
VGENVPDDLYIKGSG

SEQ ID NO: 9 - protein, 19 aa, Artificial Sequence
  Synthetic polypeptide
GLKAKPKFTLGKRKATPTT

SEQ ID NO: 10 - protein, 14 aa, Artificial Sequence
  Synthetic polypeptide
ENVPDDLYIKGSGS

SEQ ID NO: 11 - protein, 20 aa, Artificial Sequence
  Synthetic polypeptide
QPLGVGISGHPLLNKLDDTE

SEQ ID NO: 12 - protein, 20 aa, Artificial Sequence
  Synthetic polypeptide
FNRAGTVGENVPDDLYIKGS

SEQ ID NO: 13 - protein, 20 aa, Artificial Sequence
  Synthetic polypeptide
STILEDWNFGLQPPPGGTLE

SEQ ID NO: 14 - protein, 12 aa, Artificial Sequence
  Synthetic polypeptide
PCTNVAVNPGDC

SEQ ID NO: 15 - protein, 20 aa, Artificial Sequence
  Synthetic polypeptide
DVNVYHIFFQMSLWLPSEAT

SEQ ID NO: 16 - protein, 20 aa, Artificial Sequence
  Synthetic polypeptide
SSILEDWNFGVPPPPTTSLV

SEQ ID NO: 17 - protein, 21 aa, Artificial Sequence
  Synthetic polypeptide
LNKYDDVENSGGYGGNPGQDN

SEQ ID NO: 18 - protein, 6 aa, Artificial Sequence
  Synthetic polypeptide
FSIKRA

SEQ ID NO: 19 - protein, 10 aa, Artificial Sequence
  Synthetic polypeptide
KQCTNTPVQA

SEQ ID NO: 20 - protein, 20 aa, Artificial Sequence
  Synthetic polypeptide
PNNNKILVPKVSGLQYRVFR

SEQ ID NO: 21 - protein, 20 aa, Artificial Sequence
  Synthetic polypeptide
LYIKGSGSTANLASSNYFPT

SEQ ID NO: 22 - protein, 20 aa, Artificial Sequence
  Synthetic polypeptide
EDTYRFVTQAIACQKHTPPA

SEQ ID NO: 23 - protein, 19 aa, Artificial Sequence
  Synthetic polypeptide
TVGESVPTDLYIKGSGSTA

SEQ ID NO: 24 - protein, 20 aa, Artificial Sequence
  Synthetic polypeptide
AGTVGENVPDDLYIKGSGST

SEQ ID NO: 25 - protein, 20 aa, Artificial Sequence
  Synthetic polypeptide
AGTMGDTVPQSLYIKGTGMR

SEQ ID NO: 26 - protein, 20 aa, Artificial Sequence
  Synthetic polypeptide
SGTVGESVPTDLYIKGSGST

SEQ ID NO: 27 - protein, 22 aa, Artificial Sequence
  Synthetic polypeptide
AGVMGDTVPTDLYIKGTSANMR

SEQ ID NO: 28 - protein, 25 aa, Artificial Sequence
  Synthetic polypeptide
LLQAGLKAKPKFTLGKRKATPTTSS

SEQ ID NO: 29 - protein, 25 aa, Artificial Sequence
  Synthetic polypeptide
LVQAGLRRKPTIGPRKRSAPSATTS

SEQ ID NO: 30 - protein, 25 aa, Artificial Sequence
  Synthetic polypeptide
LLQAGYRARPKFKAGKRSAPSASTT

SEQ ID NO: 31 - protein, 25 aa, Artificial Sequence
  Synthetic polypeptide
LVQAGLRRRPTIGPRKRPAASTSTA

SEQ ID NO: 32 - protein, 13 aa, Artificial Sequence
  Synthetic polypeptide
LVEETSFIDAGAP

SEQ ID NO: 33 - protein, 38 aa, Artificial Sequence
  Synthetic polypeptide
LGTRPPTATDTLAPVRPPLTVDPVGPSDPSIVSLVEET

SEQ ID NO: 34 - protein, 40 aa, Artificial Sequence
  Synthetic polypeptide
IDAGAPTSVPSIPPDVSGFSITTSTDTTPAILDINNTVTT

SEQ ID NO: 35 - protein, 27 aa, Artificial Sequence
  Synthetic polypeptide
NNTVTTVTTHNNPTFTDPSVLQPPTPA

SEQ ID NO: 36 - protein, 29 aa, Artificial Sequence
  Synthetic polypeptide
PTPAETGGHFTLSSSTISTHNYEEIPMDT

SEQ ID NO: 37 - protein, 21 aa, Artificial Sequence
  Synthetic polypeptide
LYKTCKQAGTCPPDIIPKVEG

SEQ ID NO: 38 - protein, 21 aa, Artificial Sequence
  Synthetic polypeptide
CGGLGIGTGSGTGGRTGYIPL

SEQ ID NO: 39 - protein, 16 aa, Artificial Sequence
  Synthetic polypeptide
CGTGSGTGGRTGYIPL

SEQ ID NO: 40 - protein, 19 aa, Artificial Sequence
  Synthetic polypeptide
CSGTGGRTGYIPLGTRPPT

SEQ ID NO: 41 - protein, 21 aa, Artificial Sequence
  Synthetic polypeptide
CDPVGPSDPSIVSLVEETSFI

SEQ ID NO: 42 - protein, 13 aa, Artificial Sequence
  Synthetic polypeptide
RTGYIPLGTRPPT

SEQ ID NO: 43 - protein, 12 aa, Artificial Sequence
  Synthetic polypeptide
SGYIPANTTIPF

SEQ ID NO: 44 - protein, 20 aa, Artificial Sequence
  Synthetic polypeptide
PMDTFIVSTNPNTVTSSTPI

SEQ ID NO: 45 - protein, 19 aa, Artificial Sequence
  Synthetic polypeptide
KTCKQAGTCPPDIIPKVEG

SEQ ID NO: 46 - protein, 20 aa, Artificial Sequence
  Synthetic polypeptide
IIPKVEGKTIAEQILQYGSM

SEQ ID NO: 47 - protein, 20 aa, Artificial Sequence
  Synthetic polypeptide
TGSGTGGRTGYIPLGTRPPT

SEQ ID NO: 48 - protein, 20 aa, Artificial Sequence
  Synthetic polypeptide
SSTPIPGSRPVARLGLYSRT

SEQ ID NO: 49 - protein, 20 aa, Artificial Sequence
  Synthetic polypeptide
DNSINIAPDPDFLDIVALHR

SEQ ID NO: 50 - protein, 20 aa, Artificial Sequence
  Synthetic polypeptide
NNGLYDIYADDFITDTSTTP

SEQ ID NO: 51 - protein, 27 aa, Artificial Sequence
  Synthetic polypeptide
SATQLYKTCKQAGTCPPDIIPKVEGKT

SEQ ID NO: 52 - protein, 15 aa, Artificial Sequence
  Synthetic polypeptide
QLYKTCKQAGTCPPD

SEQ ID NO: 53 - protein, 17 aa, Artificial Sequence
  Synthetic polypeptide
GTGGRTGYIPLGTRPPT